SEQUENCE LISTING (18 sequences)

SEQ ID NO: 1 - DNA, 24 bp, synthetic construct
  source 1..24: mol_type = other DNA; note = primer
ccctggggatgctgtgagccaagg

SEQ ID NO: 2 - DNA, 24 bp, synthetic construct
  source 1..24: mol_type = other DNA; note = primer
ccctttttctggagactaaataaa

SEQ ID NO: 3 - DNA, 24 bp, synthetic construct
  source 1..24: mol_type = other DNA; note = primer
gcgaactcacacaggcgagaaacc

SEQ ID NO: 4 - DNA, 24 bp, synthetic construct
  source 1..24: mol_type = other DNA; note = primer
ttatcgtcgaccactgtgctgctg

SEQ ID NO: 5 - DNA, 24 bp, synthetic construct
  source 1..24: mol_type = other DNA; note = primer
ggttacctcttcctcccactccag

SEQ ID NO: 6 - DNA, 24 bp, synthetic construct
  source 1..24: mol_type = other DNA; note = primer
cagaggaggaacgagctgaagcgc

SEQ ID NO: 7 - DNA, 21 bp, synthetic construct
  source 1..21: mol_type = other DNA; note = primer
attgagatgagaaccggcatg

SEQ ID NO: 8 - DNA, 21 bp, synthetic construct
  source 1..21: mol_type = other DNA; note = primer
ttcaacatcactgccagctcc

SEQ ID NO: 9 - DNA, 18 bp, synthetic construct
  source 1..18: mol_type = other DNA; note = primer
tgtgagcgagtaacaacc

SEQ ID NO: 10 - DNA, 20 bp, synthetic construct
  source 1..20: mol_type = other DNA; note = primer
tgactttctgtgctcagagg

SEQ ID NO: 11 - DNA, 20 bp, synthetic construct
  source 1..20: mol_type = other DNA; note = primer
caatgatggctccagggtaa

SEQ ID NO: 12 - DNA, 20 bp, synthetic construct
  source 1..20: mol_type = other DNA; note = primer
aagggactggctgctattgg

SEQ ID NO: 13 - DNA, 21 bp, synthetic construct
  source 1..21: mol_type = other DNA; note = primer
gtacacgtttctcctcaggac

SEQ ID NO: 14 - DNA, 20 bp, synthetic construct
  source 1..20: mol_type = other DNA; note = primer
atatcacgggatgccaacgc

SEQ ID NO: 15 - DNA, 21 bp, synthetic construct
  source 1..21: mol_type = other DNA; note = primer
tctccagtgtgagttctctcg

SEQ ID NO: 16 - DNA, 21 bp, synthetic construct
  source 1..21: mol_type = other DNA; note = primer
attgagatgagaaccggcatg

SEQ ID NO: 17 - DNA, 21 bp, synthetic construct
  source 1..21: mol_type = other DNA; note = primer
ttcaacatcactgccagctcc

SEQ ID NO: 18 - DNA, 18 bp, synthetic construct
  source 1..18: mol_type = other DNA; note = primer
tgtgagcgagtaacaacc

(19) United States
(12) Patent Application Publication
(10) Pub. No.: US 2026/0090528 A1
(43) Pub. Date: Apr. 2, 2026

(54) ORGAN REGENERATION METHOD UTILIZING iPS CELL AND BLASTOCYST COMPLEMENTATION

(71) Applicant: The University of Tokyo, Tokyo (JP)

(72) Inventors: Hiromitsu NAKAUCHI, Tokyo (JP); Toshihiro Kobayashi, Tokyo (JP); Tomoyuki Yamaguchi, Tokyo (JP); Sanae Hamanaka, Tokyo (JP)

(21) Appl. No.: 19/407,344

(22) Filed: Dec. 3, 2025

Related U.S. Application Data
(63) Continuation of application No. 17/412,947, filed on Aug. 26, 2021, now abandoned.

(51) Int. Cl. A01K 67/0271; A01K 67/0275; A01K 67/0276; A61L 27/38

(52) U.S. Cl. CPC A01K 67/0271; A01K 67/0275; A01K 67/0276; A61L 27/3834; A61L 27/3839; A61L 27/3895; A01K 2207/12; A01K 2227/105; A01K 2267/025

(57) ABSTRACT

It is revealed that an organ such as pancreas can be regenerated by utilizing a fact that the deficiency of an organ is complemented by injecting an induced pluripotent stem cell (iPS cell) into a developed blastocyst in a blastocyst complementation method. Thus, the present invention has solved the above-described object. This provides a method for producing a target organ, using an iPS cell, in a living body of a non-human mammal having an abnormality associated with a lack of development of the target organ in a development stage, the target organ produced being derived from a different individual mammal that is an individual different from the non-human mammal.

### STATEMENT REGARDING SEQUENCE LISTING

The instant application contains a Sequence Listing in XML format, which is hereby incorporated by reference in its entirety. Said XML copy, created on Dec. 1, 2025, is named 51890-003005_Sequence_Listing_12_1_25 and is 19,792 bytes in size.

### TECHNICAL FIELD

The present invention relates to a method for producing a desired cell-derived organ in vivo using an iPS cell.

### BACKGROUND ART

In discussing regenerative medicine in the form of cell transplantation or organ transplantation, expectations for pluripotent stem cells are high. ES cells established from the inner cell mass of blastocyst stage fertilized eggs are pluripotent, and therefore used in various studies on cell differentiation. Development of differentiation control methods of inducing differentiation of such ES cells into specific cell lineages in vitro is a topic in the field of regenerative medicine research.

In the research on in vitro differentiation using ES cells, differentiation into mesoderms and ectoderms, such as hemocytes, blood vessels, myocardia, and nervous systems, which differentiate during early embryogenesis, is likely to occur. However, there is known a general tendency that differentiation into organs directed to the formation of complicated tissues through intracellular interactions during and after the middle embryogenesis is difficult.

For example, a metanephros, which is an adult kidney of mammals, develops from intermediate mesoderm during middle embryogenesis. Specifically, the development of kidney is initiated by the interaction between two components, which are a metanephric mesenchymal cell and a ureteric bud epithelium. Finally, the adult kidney is completed through differentiations into a number of types of functional cells, which is as large as dozens and cannot be seen in other organs, and through the formation of a complicated nephron structure, which is mainly composed of a glomerulus and a renal tubule, as a result of the differentiations. It is easily inferred from the timing of kidney development and the complication of the process thereof that induction of a kidney from ES cells in vitro is an extremely labor-intensive work, and the induction is considered to be actually impossible. Further, identification of somatic stem cells in organs, such as kidney, has not been established yet, and it has started to be revealed that contribution of bone marrow cells to the repair processes of injured kidney, which was once used to be actively studied, is not very significant.

When a pluripotent ES cell is injected into the inner space of a blastocyst stage fertilized egg, a resulting individual forms a chimeric mouse. There has been previously reported a rescue experiment of T-cell and B-cell lineages by blastocyst complementation, to which this technique is applied, the rescue experiment being carried out on a Rag-2 knockout mouse deficient in T-cell and B-cell lineages (Non-Patent Document 1). This chimeric mouse assay is used as an in vivo assay system for verifying the differentiation of the T-cell lineage, for which no in vitro assay system is available.

However, even if such a technique is found to be available for a certain organ, it is difficult to predict whether the technique will actually be effective in other organs, because of the difference in the role of the organs in the living body, for example, the difference in fatality or the like resulting from the absence of the organs. Various factors also affect the validity of the technique. In addition, the deficient genes of the organ deficiency model selected in this instance are also an important factor. This is conceivably because it is required to select transcription factors that are essential for the function of the deficient genes during the development process, particularly for the differentiation and maintenance of stem/precursor cells of each organ during the process of organ formation.

It is expected that when a model representing organ deficiency caused by the deficiency of a humoral factor or a secretion factor is to be used, only the factors supposed to be released are complemented by the factors released from the ES cell-derived cells, resulting in a chimeric state at the organ level.

Accordingly, selection of an appropriate model animal for an organ is the key factor in the present invention. In considering the application to other organs, it is thought to be difficult to use a model representing the same phenotype as that of the present invention with respect to other organs.

The present inventors have filed an application PCT/JP2008/51129 as an organ regeneration method.

In addition, induced pluripotent stem (iPS) cells have recently drawn attention (for example, Non-Patent Document 2). The iPS cells are regarded to have equivalent functions to those of ES cells.

### CITATION LIST

### Non Patent Literature

NPL1: Chen J., et al., Proc. Natl. Acad. Sci. USA, Vol. 90, pp. 4528-4532, 1993
NPL2: Okita K et al., Generation of germline-competent induced pluripotent stem cells. Nature 448 (7151) 313-7, 2007

### SUMMARY OF INVENTION

### Technical Problem

An object of the present invention is to provide a technique for organ regeneration using a readily preparable induced pluripotent stem cell (iPS cell), the technique being suitable for industrial application. Specifically, the object is to provide a technique for regenerating an “own organ” from a somatic cell, such as skin, depending on the circumstance of an individual. Moreover, another object is to conduct research and development using organs derived from various genomes, the organs being provided by carrying out the present invention by way of producing an induced pluripotent stem cell (iPS cell) from a cell having a target genome. Still another object is to avoid an ethical problem that has been a problem in ES cells.

### Solution to Problem

It has been discovered that, in a blastocyst complementation method, a next generation is born when a deficiency of an organ, such as pancreas, is complemented by injection of induced pluripotent stem cells (iPS cells) into a developed blastocyst, and further discovered that a transgenic animal having the pancreas thus complemented can transmit its phenotype to the next generation as a founder. These discoveries have revealed that organ regeneration can be accomplished by using such a founder. Thus, the present invention has solved the above-described problems.

In the present invention, it has been discovered that a litter can be efficiently obtained using founders obtained by transplanting induced pluripotent stem cells (iPS cells) as pluripotent cells into knockout mice and transgenic animals (for example, mice), which are characterized by having a deficiency of organ, such as pancreas, so as to complement the pancreas.

In the present invention, it was found from the result of genotyping that even if induced pluripotent stem cells (iPS cells) are used, knockout mice each with a pancreas complemented grow to normal adults.

The complemented knockout (hereinafter, also referred to as “KO”) mouse was expected to be theoretically a KO or hetero individual at a probability of ½ according to Mendelian inheritance, as being derived from breeding between a hetero mouse and the KO which is capable of transmitting its phenotype to the next generation as a founder. This was found to be as expected in reality. From this, it is possible to obtain a KO individual at a probability of 100% in the next generation from breeding between KO individuals in which pancreas has been complemented. Therefore, it is expected that analysis using KO individuals will be able to be carried out significantly more easily.

Meanwhile, in a conventional method for producing a transgenic (Tg) animal, a transgene for inducing a deficiency of an organ is introduced into an egg cell followed by transplantation of a resulting egg cell. In a relatively new method, a next generation is born when a deficiency of pancreas is complemented by injection of ES cells into a developed blastocyst. It was revealed that in both of the methods, induced pluripotent stem cells (iPS cells) can be used. Furthermore, it was discovered that a transgenic animal with a pancreas thus complemented by use of induced pluripotent stem cells (iPS cells) is also capable of transmitting its phenotype to the next generation as a founder. Thus, it has been revealed that organ regeneration can be carried out using such a founder obtained by use of induced pluripotent stem cells (iPS cells), as well.

It should be understood that, once the method of the present invention is found to be applicable to a certain organ, appropriate modifications on the basis of previous successful cases can be applied to the organ. The reason for this is as follows. If an appropriate defective animal is available, a similar method of analysis can be applied thereto using fluorescent-labeled iPS cells (derived from fibroblast collected from a skin or tail, for example) or the like as indicated in the present description, so as to reveal whether a thus constructed organ is derived from the host or from iPS cells or the like. This allows a judgment whether organ construction has been successful or not. Thus, it should be understood in accordance with the same theory that a next generation animal can be reproduced.

Therefore, the present invention provides the followings.

In one aspect, the present invention provides a method for producing a target organ in a living body of a non-human mammal having an abnormality associated with a lack of development of the target organ in a development stage, the target organ produced being derived from a different individual mammal that is an individual different from the non-human mammal, the method comprising the steps:
a) preparing an induced pluripotent stem cell (iPS cell) derived from the different individual mammal;
b) transplanting the cell into a blastocyst stage fertilized egg of the non-human mammal;
c) developing the fertilized egg in a womb of a non-human surrogate parent mammal to obtain a litter; and
d) obtaining the target organ from the litter individual.

In one embodiment, the iPS cell is derived from any one of a human, a rat, and a mouse.

In one embodiment, the iPS cell is derived from any one of a rat and a mouse.

In one embodiment, the organ to be produced is selected from a pancreas, a kidney, a thymus, and a hair.

In one embodiment, the non-human mammal is a mouse.

In one embodiment, the mouse is any one of a Sall1 knockout mouse, a Pdx1-Hes1 transgenic mouse, a Pdx1 knockout mouse, and a nude mouse.

In one embodiment, the target organ is completely derived from the different individual mammal.

In one embodiment, the method of the present invention further comprises a step of bringing a reprogramming factor into contact with a somatic cell to obtain the iPS cell.

In one embodiment, in the method of the present invention, the iPS cell and the non-human mammal are in a xenogeneic relationship.

In one embodiment, in the method of the present invention, the iPS cell is derived from a rat, and the non-human mammal is a mouse.

In another aspect, the present invention provides a non-human mammal having an abnormality associated with a lack of development of a target organ in a development stage, the mammal being produced by a method including the steps of:
a) preparing an iPS cell derived from a different individual mammal that is an individual different from the non-human mammal;
b) transplanting the iPS cell into a blastocyst stage fertilized egg of the non-human mammal; and
c) developing the fertilized egg in a womb of a non-human surrogate parent mammal to obtain a litter.

In another aspect, the present invention relates to use of a non-human mammal having an abnormality associated with a lack of development of a target organ in a development stage, for production of the target organ using an iPS cell.

In another aspect, the present invention provides a set for producing a target organ, the set comprising:
A) a non-human mammal having an abnormality associated with a lack of development of the target organ in a development stage; and
B) any one of
   an iPS cell derived from a different individual mammal that is an individual different from the non-human mammal, and
   a reprogramming factor and, if necessary, a somatic cell.

In another aspect, the present invention provides a method for producing any one of a target organ and a target body part, the method comprising the steps of:
A) providing an animal which includes a deficiency responsible gene coding for a factor which causes a deficiency of any one of an organ and a body part and gives any one of no possibility of survival and difficulty in survival if the factor functions, and in which the any one of an organ and a body part is complemented by blastocyst complementation, the deficiency responsible gene coding for a factor which causes a deficiency of the any one of a target organ and a target body part;
B) growing an ovum obtained from the animal into a blastocyst;
C) introducing a target iPS cell into the blastocyst so as to produce a chimeric blastocyst, the target iPS cell having a desired genome capable of complementing a deficiency caused by the deficiency responsible gene; and
D) producing an individual from the chimeric blastocyst, and then obtaining the any one of a target organ and a target body part from the individual.

In one embodiment, the method of the present invention further comprises a step of bringing a reprogramming factor into contact with a somatic cell to obtain the iPS cell.

In one embodiment, the step D) includes developing the chimeric blastocyst in a womb of a non-human surrogate parent mammal to obtain a litter, and obtaining the target organ from the litter individual.

In another embodiment, the target iPS cell is derived from any one of a rat and a mouse.

In another embodiment, the any one of a target organ and a target body part is selected from a pancreas, a kidney, a thymus, and a hair.

In still another embodiment, the animal is a mouse.

In another embodiment, the mouse is any one of a Sall1 knockout mouse, a Pdx1 knockout mouse, a Pdx1-Hes1 transgenic mouse, and a nude mouse.

In still another embodiment, the any one of a target organ and a target body part is completely derived from the target pluripotent cell.

In still another embodiment, the iPS cell and the non-human mammal are in a xenogeneic relationship.

In still another embodiment, the iPS cell is derived from a rat, and the non-human mammal is a mouse.

In another aspect, the present invention provides a set for producing any one of a target organ and a target body part, the set comprising:
A) a non-human animal which includes a gene coding for a factor which causes a deficiency of any one of an organ and a body part and gives any one of no possibility of survival and difficulty in survival if the factor functions, and in which the any one of an organ and a body part is complemented by complement; and
B) any one of
   an iPS cell derived from a different individual mammal that is an individual different from the non-human mammal, and
   a combination of a reprogramming factor and, if necessary, a somatic cell.

In one embodiment, the non-human animal and the iPS cell are in a xenogeneic relationship.

In the present invention, cells to be transplanted are prepared in accordance with the species of an animal for the organ to be produced. For example, when a human organ is to be produced, cells derived from a human are prepared. When an organ of a mammal other than human is to be produced, cells derived from the mammal are prepared. In the present invention, as the cells to be transplanted, induced pluripotent stem cells (iPS cells) can be used.

The organ to be produced in the method of the present invention may be any solid organ with a fixed shape, such as kidney, heart, pancreas, cerebellum, lung, thyroid gland, hair, and thymus. Preferable examples thereof include kidney, pancreas, hair, and thymus. Such solid organs are produced in the body of a litter by developing totipotent cells or pluripotent cells within an embryo that serves as a recipient. The totipotent cells or pluripotent cells can form all kinds of organs by being developed in an embryo. Accordingly, there is no limitation to the solid organ that can be produced depending on the kind of the totipotent cells or pluripotent cells to be used.

Meanwhile, the present invention is characterized in that an organ derived only from the transplanted cells is formed in the body of a litter individual derived from non-human embryo that serves as a recipient. Thus, it is not desirable to have a chimeric cell composition of the transplanted cells and the cells derived from the recipient non-human embryo. Therefore, as the recipient non-human embryo, it is desirable to use an embryo derived from an animal which has an abnormality associated with a lack of development of the organ to be produced in a development stage, and whose offspring has a deficiency of the organ. As long as the animal develops such an organ deficiency, knockout animal having an organ deficiency as a result of the deficiency of a specific gene or a transgenic animal having an organ deficiency as a result of incorporating a specific gene may be used. Alternatively, a “founder” animal described herein may be used.

For example, when a kidney is produced as the organ, embryos of a Sall1 knockout animal having an abnormality associated with a lack of development of a kidney in the development stage (Nishinakamura, R. et al., Development, Vol. 128, p. 3105-3115, 2001), or the like, can be used as the recipient non-human embryo. Meanwhile, when a pancreas is produced as the organ, embryos of a Pdx1 knockout animal having an abnormality associated with a lack of development of a pancreas in the development stage (Offield, M. F., et al., Development, Vol. 122, p. 983-995, 1996) can be used as the recipient non-human embryo. When a cerebellum is produced as the organ, embryos of a Wnt-1 (int-1) knockout animal having an abnormality associated with a lack of development of a cerebellum in the development stage (McMahon, A. P. and Bradley, A., Cell, Vol. 62, p. 1073-1085, 1990) can be used as the recipient non-human embryo. When a lung and a thyroid gland are produced as the organ, embryos of a T/ebp knockout animal having an abnormality associated with a lack of development of a lung and a thyroid gland in the development stage (Kimura, S., et al., Genes and Development, Vol. 10, p. 60-69, 1996), or the like, can be used as the recipient non-human embryo. Moreover, embryos of a dominant negative-type transgenic mutant animal model (Celli, G., et al., EMBO J., Vol. 17 pp. 1642-655, 1998) which overexpresses the deficiency of an intracellular domain of fibroblast growth factor (FGF) receptor (FGFR), and which causes deficiencies of multiple organs such as kidney and lung, can be used. Alternatively, nude mice can be used for production of hair or thymus.

In the present invention, the non-human animal derived from the recipient embryo may be any animal other than human, such as pig, rat, mouse, cattle, sheep, goat, horse, dog, chimpanzee, gorilla, orangutan, monkey, marmoset, and bonobo. It is preferable to collect embryos from a non-human animal having a similar adult size to that of the animal species for the organ to be produced.

Meanwhile, a mammal serving as the origin of the cell that is transplanted into a recipient blastocyst stage fertilized egg and that is for formation of the organ to be produced may be either human or a mammal other than human, such as, for example, pig, rat, mouse, cattle, sheep, goat, horse, dog, chimpanzee, gorilla, orangutan, monkey, marmoset, and bonobo.

The relationship between the recipient embryo and the cell to be transplanted may be an allogeanic relationship or a xenogeneic relationship.

By transplanting the cell to be transplanted, prepared as described above, into the inner space of the recipient blastocyst stage fertilized egg, a chimeric cell mixture of the blastocyst-derived inner cell and the transplanted cell may be formed in the inner space of the blastocyst stage fertilized egg.

The blastocyst stage fertilized egg having a cell transplanted as described above is transplanted into a womb of a surrogate parent that is a pseudo-pregnant or pregnant female animal of the species from which the blastocyst stage fertilized egg is derived. The blastocyst stage fertilized egg is developed in the womb of the surrogate parent to obtain a litter. Then, the target organ can be obtained as a mammal cell-derived target organ from this litter.

Therefore, these and other advantages of the present invention will become apparent as the following detailed description is read.

### Advantageous Effects of Invention

According to the present invention, a technique for organ regeneration is provided, the technique being suitable for industrial application. This also provides a technique for regenerating an “own organ” from a somatic cell, such a skin, depending on the circumstance of an individual.

Moreover, it becomes possible to conduct research and development using organs derived from various genomes, the organs being provided by carrying out the present invention by way of producing an induced pluripotent stem cell (iPS cell) from a cell having a target genome. This can be said to be a technique which was absolutely impossible in the prior art.

Furthermore, it becomes possible to avoid a part of the ethical problem that has been a problem in ES cells by use of iPS cells, and there is also an advantage that similar effects can be obtained.

### BRIEF DESCRIPTION OF THE DRAWINGS

FIG. 1 shows a therapeutic model using a construction of a pancreas derived from an iPS cell by blastocyst complementation.

FIG. 2 a. shows a strategy for establishing GFP mouse-derived iPS cells. After establishment of GFP mouse tail tip fibroblasts (TTF), three factors (reprogramming factor) were introducing into the TFT, and resulting TFT was cultured in an ES cell medium for 25 to 30 days. Then, iPS colonies were picked up, thereby establishing iPS cell lines. b. shows photographs of the morphology of thus established iPS cells taken by a microscope equipped with a camera. The left shows a photograph of GFP-iPS cell #2, and the right shows that of #3. c. shows measurements of alkaline phosphatase activity. The iPS cells were photographed under a fluorescent microscope, and subjected to staining using an alkaline phosphatase staining kit (Vector Laboratories, Inc., Cat. No. SK-5200). From the left, a bright-field image, a GFP fluorescence image, and alkaline phosphatase staining are shown. d. shows identification of the introduced three factors (reprogramming factors) by PCR on genomic DNA. It is the result obtained from PCR performed on the genomic DNA extracted from the iPS cells. From the top, expressions of Klf4, Sox2, Oct3/4, c-Myc, and Myog genes are shown. From the left, results of GFP-iPS cells #2 and #3, Nanog-iPS (for four factors), and ES cell (NC) as a control are shown. At the very right, a result of distilled water is shown. Insertion of the three factors in the iPS cells used in the present invention was confirmed. e. shows analysis of an ES cell-specific gene expression pattern in the cells used in the present invention and confirmation of the expression of the introduced genes, using RT-PCR. From the top, expressions of Klf4, Sox2, Oct3/4, c-Myc, Nanog, Rex1, Gapdh genes are shown. At the bottom, a negative control (RT(−)) is shown. As for Klf4, Sox2, and Oct3/4, the expressions were confirmed each for Total RNA and transgenic (Tg). From the left, expressions of GFP-iPS cells #2 and #3, ES cell (NC) as a control, and TTF (negative control) as another control are shown. At the very right, a result of distilled water is shown. f. shows production of a chimeric mouse using the iPS cells. A result of the production of a chimeric mouse is shown, the production being performed by injecting the established iPS cells into a blastocyst obtained from breeding C57BL6 and BDF1 mouse strains. In the upper part, a bright-field image (left) and a GFP fluorescence image (right) of the mouse on embryonic day 13.5 are shown. In the lower part, an image of the mouse in the neonatal period is shown. What denoted by NC is a negative control.

FIG. 3 shows the morphologies of pancreases (5 days after birth) constructed by blastocyst complementation. While the border of the pancreas of the homo mouse is neatly made up of GFP-positive cells, that of the pancreas of the hetero mouse is chimeric, which can be observed as a dotted line.

FIG. 4 shows histological analysis of pancreases derived from iPS cells (5 days after birth). Here, frozen section samples of pancreases derived from iPS cells were prepared, subjected to nuclear staining with DAPI and an anti-GFP antibody and with an anti-insulin antibody, and then observed and photographed using an upright fluorescent microscope and a confocal laser microscope. From the left, bright-field images and GFP+DAPI images are shown, and staining with the anti-insulin antibody is shown on the right. The upper panels show Pdx1^{LacZ/LacZ} of the present invention into which GFP-iPS cells had been introduced, and the lower panels show Pdx1^{wt/LacZ} as a control into which GFP-iPS cells had been introduced.

FIG. 5 shows an experiment for confirming the presence of cells that becomes GFP negative by silencing. Bone marrow cells were collected from the mouse shown in FIG. 3, isolating hematopoietic stem/precursor cells (c-Kit+, Sca-1+, Linage marker−:KSL cells) that were found to be GFP− by a flow cytometer, and thus isolated cells were dropped onto a 96-well plate one by one. The cells were cultured under the condition of cytokine addition for 12 days to allow formation of colonies. Genomic DNA was extracted from these colonies, and used for genotyping. This enables clonal genotyping on a single cell even if cells whose GFP expression is blocked by the gene silencing are included on the GFP− side. A host cell and a cell subjected to gene silencing can be conveniently discriminated. a. shows a strategy for a colony formation method using KSL cells isolated from bone marrow cells. b. shows the morphology of hemocyte colonies on day 12 after culture. c. shows genotyping of a chimeric individual using DNA extracted from each colony. The panels in a show, from the left, a FACS pattern of the hematopoietic stem/precursor cells, c-Kit+, Sca-1+, Linage− (KSL), in the bone marrow. Photographs in b shows, from the left, the colony on day 12 after culture, a bright-field image in the center, and a GFP fluorescence image on the left. c shows a result of genotyping performed by a PCR method on DNA extracted from a colony derived from a single cell by the above-described method using a kit of Qiagen Co., Ltd. The PCR method was carried out using the same primers and conditions as those at the time of Pdx1 litter determination.

FIG. 5A shows transplantation of iPS-derived pancreatic islets into STZ-induced diabetic mice. a and b show isolation of the pancreatic islets. The iPS-derived pancreas was perfused via the common bile duct (arrow in a.) with collagenase. After density-gradient centrifugation, iPS-derived pancreatic islets that express EGFP were concentrated (b). c shows the kidney film two months after the transplantation of the pancreatic islets. A spot (arrow) where EGFP was expressed is the transplanted pancreatic islet. d shows HE staining (left panel) and GFP staining with DAPI (right panel) performed on a kidney section. e shows transplantation of 150 iPS-derived pancreatic islets into STZ-induced diabetic mice. Arrows indicates the time when an antibody cocktail (anti-INF-γ, anti-TNF-α, anti-IL-1β) was administered. The blood glucose level in the intraperitoneal cavity was measured every one week until two months elapsed after the transplantation. The STZ-induced diabetic mice into which the iPS-pancreatic islets were transplanted were represented by ▴ (black triangles) (n=6), while STZ-induced diabetic mice into which no iPS-pancreatic islets were transplanted were represented by ▪ (black squares). f shows a glucose tolerance test (GTT) performed two months after the transplantation of the pancreatic islets.

FIG. 6 shows regeneration of kidney by Blastocyst Complementation in Sall1 knockout mice. A result from genotyping of the Sall1 allele is shown in the upper part. It is understood that the mouse #3 was a Sall1 homo KO mouse. On the lower part, the morphology of the kidney (1 day after birth) regenerated by performing blastocyst complementation using iPS cells in the mouse #3 as a host. It is understood that the whole kidney in the homo KO mouse is neatly made up of GFP-positive cells. It has been revealed that it is possible to produce a kidney derived from iPS cells using a Sall1 knockout mouse.

FIG. 7 shows a photograph confirming that hairs grew on chimera mice born after blastocyst complementation was performed using B6-derived iPS cells. #1 is a C57BL/6(B6) wild type (control) mouse, and black hair is seen. #3 is a KSN nude mouse (control) and does not have hair. #2, 4 and 5 indicate three chimera mice thus obtained, and these individuals have hairs growing.

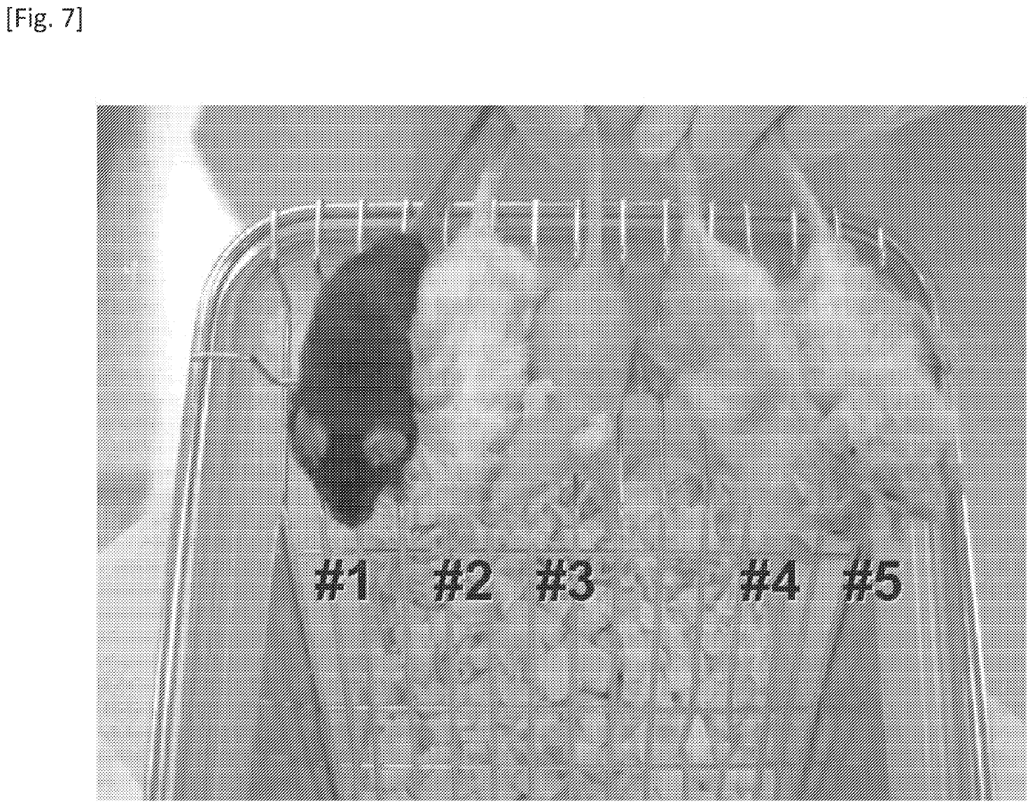

FIG. 8 shows photographs confirming the development of thymi in chimera and control mice. The thymus is observed in the C57BL/6 (B6) wild type mouse (control). A nude mouse does not have a thymus. Meanwhile, the thymus is observed in the chimeric mouse.

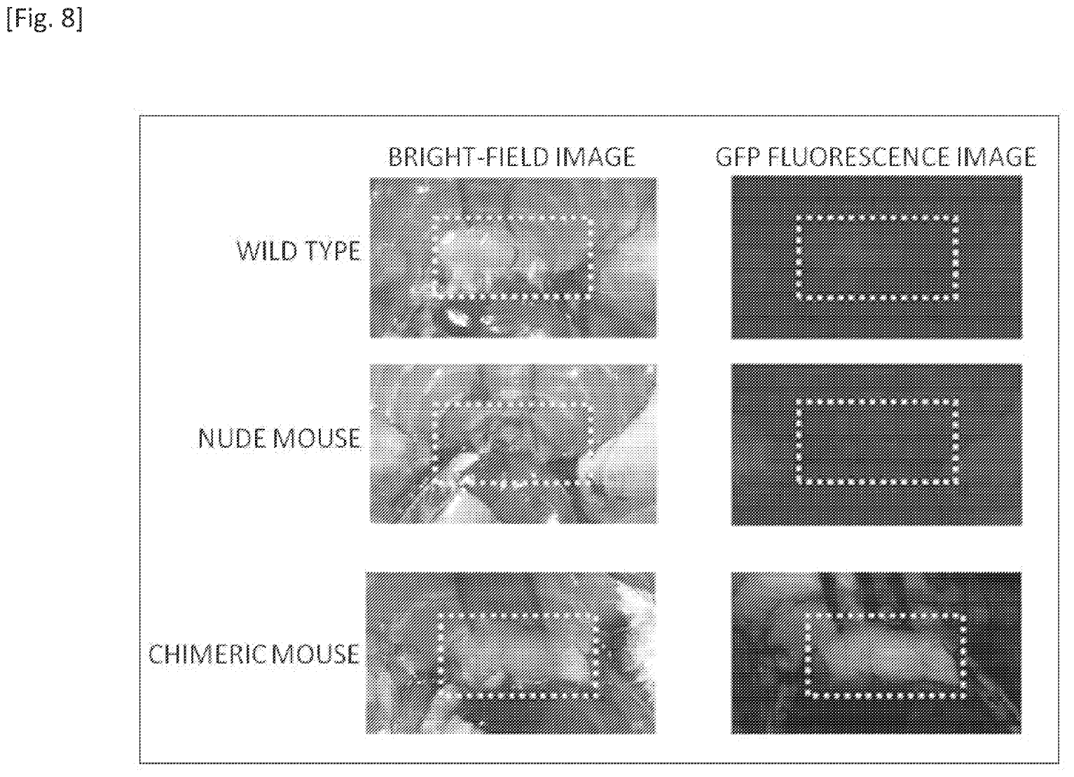

FIG. 9 shows a result of analyzing GFP-positive cells obtained from CD4- and CD8-positive cells (T cells) that were separated from peripheral blood of each of the C57BL/6 (B6) wild type (control) mouse and the chimera mice (#2, 4, and 5) in FIG. 7. The degree of chimerism is indicated from the distributions of GFP-negative cells and GFP-positive cells.

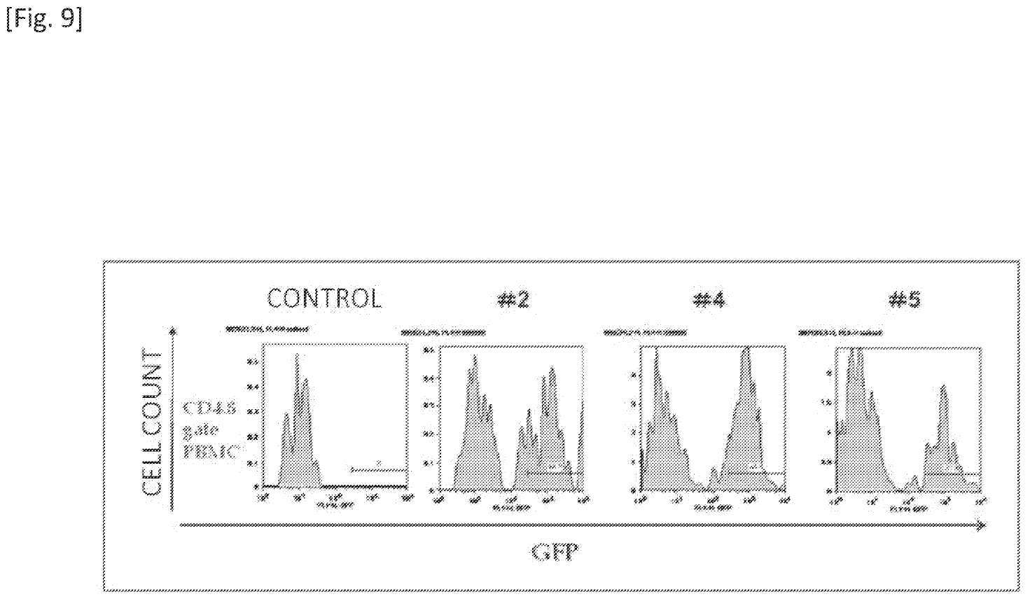

FIG. 10 A male Pdx1 (−/−) mouse (founder: which was a Pdx1 (−/−) mouse having a pancreas complemented using mouse iPS cells) was bred with a female Pdx1 (+/−) mouse. Fertilized eggs were collected and developed to the blastocyst stage in vitro. The resultant blastocyst was microinjected under a microscope with 10 rat iPS cells marked with EGFP. This was transplanted into a pseudo-pregnant surrogate parent. Laparotomy was performed in the full term pregnancy. A result of analysis of neonates thus born is shown. EGFP fluorescence was observed under a fluorescent stereoscopic microscope. It was found out from the EGFP expression on the body surface that individual numbers #1, #2, and #3 were chimeras. By laparotomy, pancreases uniformly expressing EGFP were observed in #1 and #2. Meanwhile, the pancreas of #3 exhibited partial EGFP expression, however, in a mosaic manner. Although #4 was a litter-mate as #1 to 3, no EGFP fluorescence was observed on the body surface. Because the pancreas was deficient upon laparotomy, #4 was a non-chimeric Pdx1 (−/−) mouse. Further, the spleens were removed from these neonates, and hemocyte cells prepared therefrom were subjected to staining with a monoclonal antibody against mouse or rat CD45, and analyzed by a flow cytometer. As a result, in the individual numbers #1 to 3, rat CD45-positive cells were observed in addition to mouse CD45-positive cells. Thus, it was confirmed that these were xenogeneic chimeric individuals between mouse and rat containing cells derived from the host mouse and the rat iPS cells. Furthermore, almost all the cells in the rat CD45-positive cell fractions exhibited EGFP fluorescence. Thus, the rat CD45-positive cells were cells derived from the rat iPS cells marked with EGFP.

FIG. 10A shows confirmation of the Pdx1 genotype by PCR of the host mouse of the individual numbers #1 to #3. In order to confirm the genotype of the host mouse, mouse CD45-positive cells, which are encompassed by dotted square lines in FIG. 10, were collected from the same spleen sample as in FIG. 1. The genomic DNA was extracted, and PCR was carried out using primers which are capable of distinguishing Pdx1 mutant allele and wild type allele. As a result, in #1 and #2, only mutant bands were observed, and in the individual number #3, both bands of mutant and wild type were detected. Accordingly, it is understood that the genotype of the host is Pdx1 (−/−) for #1, #2 and Pdx1 (+/−) for the individual number #3. From this result, a pancreas of rat was successfully constructed in a mouse individual by applying the xenogeneic blastocyst complementation technique using the rat iPS cells as a donor in the Pdx1 (−/−) mice #1 and #2 which should not originally have pancreases formed.

### DESCRIPTION OF EMBODIMENTS

Hereinafter, the present invention will be described. It should be understood throughout the present description that expression of a singular form includes the concept of its plurality unless otherwise mentioned. Accordingly, it should be understood that articles (for example, “a,” “an,” “the,” and the like, in English) for a singular form also include the concept of their plurality unless otherwise mentioned. It should also be understood that the terms as used herein have definitions typically used in the art unless otherwise mentioned. Thus, unless otherwise defined, all technical terms and scientific terms as used herein have the same meanings as those generally understood by those skilled in the art to which the present invention pertains. If there is contradiction, the present description (inclusive of the definition) takes precedence.

In order to specifically describe embodiments of the present invention, exemplary embodiments will be described hereinafter. As an example, a method for producing a kidney derived from a mammal cell in a living body of a mouse will be described hereinbelow. It is understood that a pancreas, a hair, and a thymus can also be produced by such a method.

### (Non-Human Animal)

In order to produce a kidney derived from a cell of a mammal other than human in a living body of an animal such as a mouse, prepared is an animal such as a mouse having an abnormality associated with a lack of development of the kidney in a development stage. In one embodiment of the present invention, a Sall1 knockout mouse (Nishinakamura, R. et al., Development, Vol. 128, p. 3105-3115, 2001) can be used as the mouse having an abnormality associated with a lack of development of the kidney in a development stage. If this animal has a homozygous knockout genotype of Sall1 (−/−), the animal is characterized in that only the kidney does not develop, and litter individuals have no kidney. Alternatively, a founder animal described herein can also be used.

This mouse has no kidney formed and cannot survive if the deficiency of Sall1 gene is in a homozygous state (Sall1 (−/−)). Thus, the deficiency of Sall1 gene is maintained in a heterozygous state (Sall1 (+/−)). Mice each in the heterozygous state are bred with each other (Sall1 (+/−)×Sall1 (+/−)), and fertilized eggs are collected from the womb. The fertilized eggs develop at a probability ratio of Sall1 (+/+):Sall1 (+/−):Sall1 (−/−)=1:2:1, in terms of probability. In the present invention, an embryo of Sall1 (−/−), which develops at a probability of 25%, is used. However, it is difficult to determine the genotype in the stage of early embryo, and thus, it is practical to determine the genotype of a litter after birth and to use only individuals having the desired genotype of Sall1 (−/−) in the subsequent steps.

This knockout mouse may have the Sall1 gene knocked out in the preparation stage and have a gene of a fluorescent protein for detection, or green fluorescent protein (GFP), knocked in into the Sall1 gene region in an expressible state (Takasato, M. et al., Mechanisms of Development, Vol. 121, p. 547-557, 2004). When the regulatory region of this gene is activated by knocking-in such a fluorescent protein, expression of GFP occurs instead of Sall1, and the deficiency state of the Sall1 gene can be determined by fluorescence detection.

Further, the relationship between a recipient embryo and a cell to be transplanted in the present invention may be an allogeanic relationship or a xenogeneic relationship. There have been hitherto a large number of reports on the preparation of a chimeric animal in such a xenogeneic relationship in the art. For example, there have been actually reported about blastular chimeric animals between closely related animal species, such as the preparation of a chimera between rat and mouse (Mulnard, J. G., C. R. Acad. Sci. Paris. 276, 379-381 (1973); Stern, M. S., Nature. 243, 472-473 (1973); Tachi, S. & Tachi, C. Dev. Biol. 80, 18-27 (1980); Zeilmarker, G., Nature, 242, 115-116 (1973)), and the preparation of a chimera between sheep and goat (Fehilly, C. B., et al., Nature, 307, 634-636 (1984)). Therefore, in the present invention, for example, in the case of preparing a kidney derived from a cell of a mammal other than human in a living body of a mouse, a certain xenogeneic organ may be prepared in a recipient embryo based on these conventionally-known chimera creation methods (for example, a method of inserting cells to be transplanted into a recipient blastocyst (Fehilly, C. B., et al., Nature, 307, 634-636 (1984))).

The term “non-human mammal” as used herein refers to a counterpart mammal from which a chimeric animal, a chimeric embryo, or the like is produced using a cell to be transplanted.

The term “different individual mammal” as used herein refers to any mammal that is an individual different from the non-human mammal, and may be an allogeanic individual or xenogeneic.

The term “non-human surrogate parent mammal” as used herein refers to a mammal in which a fertilized egg formed by transplanting a cell derived from a different individual mammal that is an individual different from a non-human mammal is developed in a womb of the non-human surrogate parent mammal (serving as a surrogate parent).

Note that although the terms “non-human mammal” and “non-human surrogate parent mammal” are sometimes referred to as a “non-human host mammal” or “host,” the “non-human mammal” and the “non-human surrogate parent mammal” are animals different from each other. In the context of the present invention, it should be understood that which is indicated is apparent to those skilled in the art.

When a pancreas is produced as the organ, embryos of a Pdx1 knockout animal having an abnormality associated with a lack of development of pancreas in a development stage (Offield, M. F., et al., Development, Vol. 122, p. 983-995, 1996) or a founder animal described herein can be used as the recipient non-human embryo.

When a hair is produced as the organ, embryos of a hairless nude mouse can be used as the recipient non-human embryo.

When a thymus is produced as the organ, embryos of a nude mouse can be used as the recipient non-human embryo.

### (Cell to be Transplanted)

Next, a cell to be transplanted into, for example, a kidney will be described. In order to produce a kidney derived from a mammal cell, an iPS cell (see Non-Patent Document 2 and so forth) or the like is prepared as the cell to be transplanted. With respect to the Sall1 gene, the cell has a wild type genotype (Sall1 (+/+)), and has an ability to develop into all kinds of cells in the kidney.

This cell may incorporate a fluorescence protein for specific detection in an expressible state prior to transplantation. For example, as a fluorescent protein used for such detection, the sequence of DsRed. T4 (Bevis B. J. and Glick B. S., Nature Biotechnology Vol. 20, p. 83-87, 2002), which is a DsRed genetic mutant, may be designed so as to be expressed in organs of almost the entire body under the control of a CAG promoter (cytomegalovirus enhancer and chicken actin gene promoter), and then be incorporated into an iPS cell by electroporation. As such a fluorescence protein, one known in the art, such as a green fluorescence protein (GFP), may be used. By performing a fluorescent labeling on such a cell for transplantation, it can be easily detected whether or not a produced organ is composed of transplanted cells only.

This mouse iPS cell or the like is transplanted into the inner space of a blastocyst stage fertilized egg having the aforementioned genotype of Sall1 (−/−) to prepare a blastocyst stage fertilized egg having a chimeric inner cell mass. This blastocyst stage fertilized egg having a chimeric inner cell mass is developed in a womb of a surrogate parent to obtain a litter. In the case of using an iPS cell which is not marked, the cell cannot be distinguished from the embryos of the host when used in the production of chimera, and it cannot be discriminated whether the complementation of the organ has been achieved. Therefore, in order to solve the problem, a fluorescent dye can be introduced into this cell line, thereby being capable of carrying out an experiment with the same protocol as those described in Examples and the like.

### (Method for Producing Founder Animal for Reproduction)

A founder animal for reproduction, used in the present invention, has the following characteristics: the animal includes a gene coding for a factor which causes a deficiency of any one of an organ and a body part and gives any one of no possibility of survival and difficulty in survival if the factor functions, and in which the any one of an organ and a body part is complemented by blastocyst complementation. By producing a next generation animal using this animal (also referred to as a “founder animal” herein), it is possible to cause a target organ to be deficient, and to produce an organ having a desired genome type regarding the deficient organ. Moreover, it has been revealed that production using this method enables organ production in the next generation as well, and also that the method can be used with iPS cells. Thus, there has been a big breakthrough in industrial application of the present invention.

The term “any one of an organ and a body part, giving any one of no possibility of survival and difficulty in survival if the factor functions” as used herein refers to, in regard to a certain factor, one that gives any one of no possibility of survival and difficulty in survival when the factor causes the any one of an organ and a body part to be deficient or dysfunctional (for example, to be not normal). For example, in the case of a foreign gene, when the gene is introduced into an animal and expressed normally, a deficiency occurs in a certain organ or body part, resulting in the animal being incapable of survival or having difficulty in survival. Difficulty in survival includes incapability of procreation of the next generation, and difficulty in the social life in a case of human. Such an organ or body part may be, for example, pancreas, liver, hair, thymus, or the like, but is not limited thereto.

Examples of genes involved in such events include Pdx-1 (for pancreas) and the like.

Incidentally, to be used for organ regeneration, a gene should be selected with which an organ can be complemented and a resulting litter does not die after birth due to other factors (being incapable of ingesting milk from a mother mouse, for example). One example of such a gene is Pdx-1. By using a gene possessing such properties, the invention of the present application can be carried out. In addition, even with the same phenotype of, for example, pancreatic deficiency, significance largely varies. Specifically, a knockout individual has a feature of improving productivity, while a transgenic individual has a feature of enabling clonal analysis of a lethal phenotype in addition to the feature of improving productivity.

The term “giving any one of no possibility of survival and difficulty in survival if the factor functions” as used herein refers to, regarding a certain factor, a condition in which, if the factor functions, an animal as a host cannot survive at all and dies, or can survive but is substantially impossible to survive later due to reasons, such as difficulties in growth and reproduction. The term can be understood by using ordinary knowledge in the art.

The term “organ” as used herein is used to have an ordinary meaning in the art, and refers to organs constituting animal viscera in general.

The term “body part” as used herein refers to any part of a body, and also includes ones which are not generally referred to as organs. For example, when a kidney is taken as an example, a complete kidney is created when genes are normal. However, when some gene is deficient or has an abnormality, although an organ like a kidney may be created, a part of the organ may have an abnormality or deficiency. The part having such an abnormality or deficiency can be said to be an example of this “body part.” Gene defect or abnormality does not necessarily correspond to each organ, and it frequently occurs that a part thereof is affected. Accordingly, when a correspondence relationship to a gene is to be considered, it may be better to consider correspondence to a body part. Therefore, such a correspondence relationship is also taken into consideration herein.

The term “blastocyst complementation” as used herein refers to a technique for complementing a defective organ or body part by using the phenomenon in which a resulting individual obtained from injection of pluripotent cells, such as ES cells and iPS cells, having multipotency into an inner space of a blastocyst stage fertilized egg forms a chimeric mouse. The inventors have discovered, regarding blastocyst complementation which had been considered to be difficult, that a mammalian organ, such as kidney, pancreas, hair, and thymus, having a complicated cellular constitution formed of multiple kinds of cells can be produced in the living body of an animal, particularly, a non-human animal. The inventors confirmed that blastocyst complementation can be carried out using iPS cells. Thus, this technique can be utilized in full scale in the present invention using iPS cells.

The term “label” as used herein may be any factor as long as it is used for distinguishing a complemented organ. For example, by causing a specific gene (such as, for example, a gene for expressing a fluorescence protein) to be expressed only in an organ to be complemented, the organ to be complemented can be distinguished from a host of complementation by a property (for example, fluorescence) derived from the specific gene. As described above, it can be distinguished whether an animal became complete by complementation with cells derived from exogenous cells or an animal became complete by complementation with cells derived from endogenous cells. Thus, it is possible to select a founder animal used in the present invention more easily. These cells may incorporate a fluorescence protein for specific detection in an expressible state prior to transplantation. For example, as a fluorescent protein used for such detection, the sequence of DsRed. T4 (Bevis B. J. and Glick B. S., Nature Biotechnology Vol. 20, p. 83-87, 2002), which is a DsRed genetic mutant, may be designed so as to be expressed in organs of almost the entire body under the control of a CAG promoter (cytomegalovirus enhancer and chicken actin gene promoter), and then be incorporated into an iPS cell by electroporation. By performing a fluorescent labeling on such a cell for transplantation, it can be easily detected whether or not a produced organ is composed of transplanted cells only.

Examples of such label include: green fluorescent protein (GFP) genes; red fluorescent proteins (RFP); cyan fluorescent proteins (CFP); other fluorescent proteins; LacZ; and the like.

A method for producing a founder animal used in the present invention includes the following steps of: A) providing a first pluripotent cell having the gene; B) growing the first pluripotent cell into a blastocyst; C) introducing a second pluripotent cell into the blastocyst so as to produce a chimeric blastocyst, the second pluripotent cell having an ability to complement a deficiency caused by the gene; and D) producing individuals from the chimeric blastocyst, and then selecting an individual in which the any one of an organ and a part thereof has been complemented by the second pluripotent cell.

The terms “(deficiency responsible) gene coding for a factor which causes a deficiency of any one of an organ and a body part and gives any one of no possibility of survival and difficulty in survival if the factor functions” and “deficiency responsible gene” as used herein are used interchangeably and refers to, in regard to a certain gene, a gene that gives any one of no possibility of survival and difficulty in survival when the factor functions (for example, in the case of a foreign gene, when the gene is introduced and expressed; in the case of an intrinsic gene, when such a gene is exposed to a condition in which the gene functions; or other cases) to cause the any one of an organ and a body part to be deficient or dysfunctional (for example, to be not normal).

Examples of “pluripotent cell” used herein include: an egg cell; an embryonic stem cell (ES cell); an induced pluripotent cell (iPS cell); a multipotent germ stem cell (mGS cell); and the like.

The term “first pluripotent cell” as used herein refers to a pluripotent cell used as an origin to be a host such as a founder animal (also referred to as a host herein) or to a cell mass derived therefrom. Preferably, a fertilized egg or an embryo is used.

The term “second pluripotent cell” when used herein refers to a pluripotent cell used with a view of an organ to be produced, and an iPS cell is used.

The term “having an ability to complement a deficiency” as used herein refers to, in regard to a factor, gene, or the like, an ability capable of complementing an organ or a body part.

The term “chimeric blastocyst” as used herein refers to a blastocyst formed by a cell, which is derived from the first pluripotent cell, and a cell, which is derived from the second pluripotent cell, being in a chimeric state. Such a chimeric blastocyst can be produced by, in addition to an injection method, utilizing a method such as a so-called “agglutination method” in which embryo+embryo, or embryo+cell are closely attached to each other in a Petri dish to produce a chimeric blastocyst. Further, the relationship between a recipient embryo and a cell to be transplanted in the present invention may be an allogeanic relationship or a xenogeneic relationship. There have been hitherto a large number of reports on the preparation of a chimeric animal in such a xenogeneic relationship in the art. For example, there have been actually reported about blastular chimeric animals between closely related animal species, such as the preparation of a chimera between rat and mouse (Mulnard, J. G., C. R. Acad. Sci. Paris. 276, 379-381 (1973); Stern, M. S., Nature. 243, 472-473 (1973); Tachi, S. & Tachi, C. Dev. Biol. 80, 18-27 (1980); Zeilmarker, G., Nature, 242, 115-116 (1973)), and the preparation of a chimera between sheep and goat (Fehilly, C. B., et al., Nature, 307, 634-636 (1984)). Therefore, in the present invention, for example, in the case of preparing a kidney derived from a cell of a mammal other than human in a living body of a mouse, a certain xenogeneic organ may be prepared in a recipient embryo based on these conventionally-known chimera creation methods (for example, a method of inserting cells to be transplanted into a recipient blastocyst (Fehilly, C. B., et al., Nature, 307, 634-636 (1984))).

In the method for producing a founder animal used in the present invention, the step of providing the first pluripotent cell having the gene coding for a factor which causes a deficiency of any one of an organ and a body part and gives any one of no possibility of survival and difficulty in survival if the factor functions (the gene also refers to as the “deficiency responsible gene” herein) can be carried out, for example, by procuring a pluripotent cell having the gene, or by producing a pluripotent cell having the gene by introducing the gene into the pluripotent cell. A method of such gene introduction is well known in the art, and those skilled in the art can carry out such gene introduction by appropriately selecting a method. It is preferable to use electroporation. In electroporation, an electric pulse is applied to a cell suspension to create fine pores on a cell membrane, and DNA is sent into the cell so that transformation, that is, introduction of a target gene can be achieved. Accordingly, damage after electroporation is small. This is why electroporation is preferable, but the method is not limited thereto.

In the method for producing a founder animal used in the present invention, the step of growing the first pluripotent cell (for example, a fertilized egg, an embryo, or the like) into a blastocyst can be carried out by any publicly-known method for growing a pluripotent cell into a blastocyst. The conditions for this are well known in the art, and described in Manipulating the Mouse Embryo, A LABORATORY MANUAL 3^{rd} Edition 2002 (Cold Spring Harbor Laboratory Press, Cold Spring Harbor, New York) (incorporated herein by reference).

In the method for producing a founder animal used in the present invention, the step of introducing an induced pluripotent stem cell (iPS cell), which is the second pluripotent cell having an ability to complement a deficiency caused by the gene, into the blastocyst so as to produce a chimeric blastocyst may adopt any publicly-known method in the art as long as the induced pluripotent stem cell (iPS cell) as the second pluripotent cell can be introduced into the blastocyst. Examples of such a method include an injection method and agglutination; however, the method is not limited to these.

In the method for producing a founder animal used in the present invention, a method for producing individuals from the chimeric blastocyst may adopt a publicly-known technique in the art. Generally, the chimeric blastocyst is returned to a surrogate parent, and then pseudo-pregnancy of the surrogate parent is caused so as to grow resulting individuals in the womb of the surrogate parent. However, the method is not limited to this technique.

In the method for producing a founder animal used in the present invention, selecting of an individual in which the any one of an organ and a body part thereof complemented can be carried out by using any technique allowing confirmation of complementation of the organ or body part.

An example thereof is identifying an identifier derived from the induced pluripotent stem cell (iPS cell) as the second pluripotent stem cell. The term “identifier” as used herein refers to any factor which allows specifying of a certain individual, species, or the like, and identifying of the origin thereof, and is also referred to as “ID” in its abbreviation. Such an identifier could be, for example, a genomic sequence, phenotype, or the like unique to the induced pluripotent stem cell (iPS cell) as the second pluripotent cell. Alternatively, regarding such selecting, by using the second pluripotent cell which is labeled or can be labeled (including one which can be a label by gene expression), the selecting in the method for producing a founder mouse of the present invention may be carried out by identifying the label. In addition, it is understood that those in the art can carry out the selecting by modifying this technique as necessary.

### (Method of Organ Regeneration Using Founder Animal)

In another aspect, the present invention provides a method for producing any one of a target organ and a target body part using a founder animal and utilizing an induced pluripotent stem cell (iPS cell). The method comprises the steps of: providing a founder animal, in which a deficiency responsible gene codes for a factor which causes a deficiency of the any one of a target organ and a target body part; B) growing an ovum obtained from the animal into an blastocyst; C) introducing an induced pluripotent stem cell (iPS cell) as a target pluripotent cell into the blastocyst so as to produce a chimeric blastocyst, the target iPS cell having a desired genome capable of complementing a deficiency caused by the gene; and D) producing an individual from the chimeric blastocyst, and then obtaining the any one of a target organ and a body part from the individual.

Here, the step D) can be carried out by developing the chimeric blastocyst in a womb of a non-human surrogate parent mammal to obtain a litter, and obtaining the target organ from the litter individual.

### (Formation of Pancreas)

The formation of a pancreas can be investigated by performing macroscopic or microscopic morphological analysis, gene expression analysis, and the like, using methods, such as visual inspection, microscopic observation after staining, and observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the pancreas.

Furthermore, the gene expression analysis using fluorescence in such a way as to emit fluorescence according to conditions may also be performed. For example, the above-described knockout mouse obtained through Pdx1-Lac-Z knock-in has the following characteristics. When a fluorescent-labeled ES cell is used in a wild type (+/+) or heterozygous (+/−) individual, mottled fluorescence in a chimeric state is shown even though the contribution of the ES cell is observed. On the other hand, in a homozygous (−/−) individual, uniform fluorescence is shown because the pancreas is constructed by a cell that is completely derived from the ES cell. Using such characteristics, it is possible to conveniently examine which genotype a target organ or a cell constituting the target organ has with respect to the Pdx1 gene. If unmarked iPS cells are used, the cells cannot be distinguished from the embryos of the host when used in the production of chimera, and it cannot be discriminated whether the complementation of the organ has been achieved. Therefore, in order to solve this problem, a fluorescent dye can be introduced into the iPS cell line, thereby being capable of carrying out an experiment with the same protocol as above. By using the cell such as described above, it is possible to produce an organ with the same protocol as the case of using the iPS cell, and to clarify the origin.

### (Formation of Kidney)

The formation of a kidney can be investigated by performing macroscopic or microscopic morphological analysis, gene expression analysis, and the like, using methods, such as visual inspection, microscopic observation after staining, and observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the kidney.

Furthermore, the gene expression analysis using fluorescence in such a way as to emit fluorescence according to conditions may also be performed. For example, the above-described Sall1 gene knockout mouse has the following characteristics. The fluorescence intensity is low when the deficiency of the Sall1 gene is in the homozygous state (Sall1 (−/−)) where GFP fluorescence occurs from both alleles, compared to the case of fluorescence when the deficiency of the Sall1 gene is in a heterozygous state (Sall1 (+/−)) where fluorescence occurs only in one allele. Using such characteristics, it is possible to conveniently examine which genotype a target organ or a cell constituting the target organ has with respect to the Sall1 gene. If unmarked iPS cells are used, the cells cannot be distinguished from the embryos of the host when used in the production of chimera, and it cannot be discriminated whether the complementation of the organ has been achieved. Therefore, in order to solve this problem, a fluorescent dye can be introduced into the iPS cell line to thereby clarify the origin.

### (Formation of Hair)

The formation of a hair can be investigated by performing macroscopic or microscopic morphological analysis, gene expression analysis, and the like, using methods, such as visual inspection and observation using fluorescence.

For example, by performing visual inspection, the actual presence or absence of a hair, and features of the hair, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the hair.

Furthermore, the gene expression analysis using fluorescence in such a way as to emit fluorescence according to conditions may also be performed. For example, in the case of the above-described nude mouse, because of strong self-fluorescence of hair, it is very difficult to determine whether the produced hair is derived from the nude mouse or from the iPS cell with the naked eye under a fluorescent microscope. However, the observation can also be performed by means for appropriately observing the fluorescence. Using such characteristics, it is possible to conveniently examine which genotype a target organ or a cell constituting the target organ has. If unmarked iPS cells are used, the cells cannot be distinguished from the embryos of the host when used in the production of chimera, and it cannot be discriminated whether the complementation of the organ has been achieved. Therefore, in order to solve this problem, a fluorescent dye can be introduced into the iPS cell line, thereby being capable of carrying out an experiment with the same protocol as above. By using such cells as described above, it is possible to produce an organ with the same protocol as the case of using the iPS cell, and to clarify the origin.

### (Formation of Thymus)

The formation of a thymus can be investigated by performing macroscopic or microscopic morphological analysis, gene expression analysis, and the like, using methods, such as visual inspection, microphotographs, FACS, and observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the thymus.

Furthermore, the gene expression analysis using fluorescence in such a way as to emit fluorescence according to conditions may also be performed. For example, the above-described nude mouse has the following characteristics. The nude mouse does not conventionally have thymus, but this does not affect the survival. Accordingly, the nude mouse is born naturally without the thymus and survives. If a fluorescent-labeled iPS cell is injected thereinto by blastocyst complementation, a large number of individuals in which the contribution of the iPS cell is confirmed have the thymus showing fluorescence. Using such characteristics, it is possible to conveniently examine which genotype a target organ or a cell constituting the target organ has.

### (iPS Cell)

iPS cells can be produced by other methods. Specifically, iPS cells can be produced by bringing a reprogramming factor (which may be a single factor or in combination of multiple factors) into contact with somatic cells so as to induce initialization. Examples of such initialization and reprogramming factor include the following. For example, in Examples of the present invention, iPS cells were uniquely produced by the inventors using 3 factors (Klf4, Sox2, and Oct3/4, which are typical “reprogramming factors” used in the present invention) and a fibroblast collected from a tail of a GFP transgenic mouse. Other combinations than this, for example, 4 factors including Oct3/4, Sox2, Klf4, and c-Myc, which are called Yamanaka factors, may also be used. A modified method thereof may also be used. It is also possible to establish iPS cells using n-Myc instead of c-Myc, and using a lentivirus vector, which is a type of retrovirus vector (Blelloch R et al., (2007). Cell Stem Cell 1: 245-247). Further, human iPS cells have been successfully established by introducing four genes, which are Oct3/4, Sox2, Nanog, and Lin28, into a fetal lung-derived fibroblast or neonatal foreskin-derived fibroblast (Yu J, et al., (2007). Science 318: 1917-1920).

It is also possible to produce human iPS cells from a fibroblast-like synoviocyte and a neonatal foreskin-derived fibroblast by using mouse genes homologous to human genes, Oct3/4, Sox2, Klf4, and c-Myc, which were used in establishing mouse iPS cells (Takahashi K, et al., (2007). Cell 131: 861-872). It is also possible to establish human iPS cells by using six genes which are hTERT and SV40 large T in addition to the four genes including Oct3/4, Sox2, Klf4, and c-Myc (Park I H, et al., (2007). Nature 451: 141-146). Further, although at a low efficiency, establishment of iPS cells in mouse and human by only using 3 factors, Oct-4, Sox2, and Klf4, without introduction of the c-Myc gene has been indicated to be possible. Since the iPS cells are successfully prevented from turning into cancer cells, these can also be used in the present invention (Nakagawa M, et al., (2008). Nat Biotechnol 26: 101-106.; Wering M, et al., (2008). Cell Stem Cell 2: 10-12).

The target organ obtained according to the present invention is characterized by being completely derived from the different individual mammal. In a conventional method, a chimera was regenerated. While not wishing to be bound by theory, it is conceivable that this is because the transcription factor is necessary to the functions of the deficient gene during the development process, particularly to the differentiation and maintenance of the stem/precursor cells of each organ during the process of the formation of the organ. iPS cells can be used, and the production of iPS cells is as described above. Note that, in the case of an iPS cell line called Nanog-iPS, since the iPS cell line is not marked, the cells cannot be distinguished from the embryos of the host when used in the production of chimera, and it cannot be discriminated whether the complementation of organ has been achieved. Therefore, in order to solve this problem, a fluorescent dye can be introduced into this Nanog-iPS cell line, thereby being capable of carrying out an experiment with the same protocol as the case of using the ES cell. If the cell such as described above is used, it is possible to produce an organ with the same protocol as the case of using the ES cell, and to clarify the origin.

The present invention also provides a mammal produced by the method of the present invention. It is considered that the animal itself is also valuable as an invention because such an animal having a target organ could not be produced in the past. While not wishing to be bound by theory, it is conceivable that the reason why such an animal could not be produced in the past is because the deficient organ due to the gene deficiency was necessary for survival, and there was no way to rescue them.

Furthermore, the present invention also provides use of a non-human mammal having an abnormality associated with a lack of development of a target organ in a development stage, for production of the target organ. Use of a host cell for such a use was not sufficiently assumed in the past. Accordingly, it is considered that the animal itself is also valuable as an invention. While not wishing to be bound by theory, it is conceivable that the reason why such animals could not be produced in the past is because the deficient organ due to the gene deficiency was necessary for survival and it was impossible to maintain a target individual to sexual maturity.

(Points to Remember when Using Various Animals)

The cases of using animals other than a mouse can be performed by applying a technique described in Examples herein upon paying attention to the following points. For example, regarding the production of a chimera in other species of animals, specifically in species other than mice, there are more reports of chimeras into which an embryo or an inner cell mass, which is a part of an embryo and is an origin of an ES cell, is injected, than reports of establishment of pluripotent stem cells having an ability to form a chimera (rat: (Mayer, J. R. Jr. & Fretz, H. I. The culture of preimplantation rat embryos and the production of allophenic rats. J. Reprod. Fertil. 39, 1-10 (1974)); cattle: (Brem, G. et al. Production of cattle chimerae through embryo microsurgery. Theriogenology. 23, 182 (1985)); pig: (Kashiwazaki N et al., Production of chimeric pigs by the blastocyst injection method, Vet. Rec. 130, 186-187 (1992)). However, even when a chimera into which an inner cell mass is injected is used, the method described herein may be applied. By using an inner cell mass as described above, it is substantially possible to complement a deficient organ of a defected animal. In other words, for example, the above-described cells are each cultivated to grow into a blastocyst in vitro, a portion of inner cell mass is physically separated from thus obtained blastocyst, and then, the portion may be injected into a blastocyst. A chimeric embryo can be produced by agglutinating the 8 cell-stage ones or morulas in mid-course.

### (General Techniques)

The molecular biological method, the biochemical method, and the microbiological method used herein are well known and commonly used in the art, and are disclosed in, for example: Sambrook J. et al. (1989). Molecular Cloning: A Laboratory Manual, Cold Spring Harbor, and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press; separate-volume laboratory medicine “Experimental technique for gene transfer & expression analysis” Yodosha, 1997; and so on. The parts (or could be all) of these documents related to the present description are incorporated herein by reference.

A DNA synthesis technique and nucleic acid chemistry for producing an artificially synthesized gene are disclosed in, for example: Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approac, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press; and so on. The parts of these documents related to the present description are incorporated herein by reference.

Reference documents cited herein, such as science documents, patents, and patent applications, are incorporated herein by reference in their entirety to an extent that each of which is specifically described.

The preferred embodiments have been described for easy understanding of the present invention. Hereinafter, the present invention will be described based on examples; however, the above description and the following examples are provided only for exemplary purposes and are not provided for the purpose of limiting the present invention. Therefore, the scope of the present invention is not limited to the embodiments or examples which are specifically described herein, and is limited only by the claims.

### EXAMPLES

In the present examples, the following experiments were carried out in compliance with the regulations established in Tokyo University for the handling of animals with the spirit of kindness to animals.

### (Example of Preparation of iPS Cell)

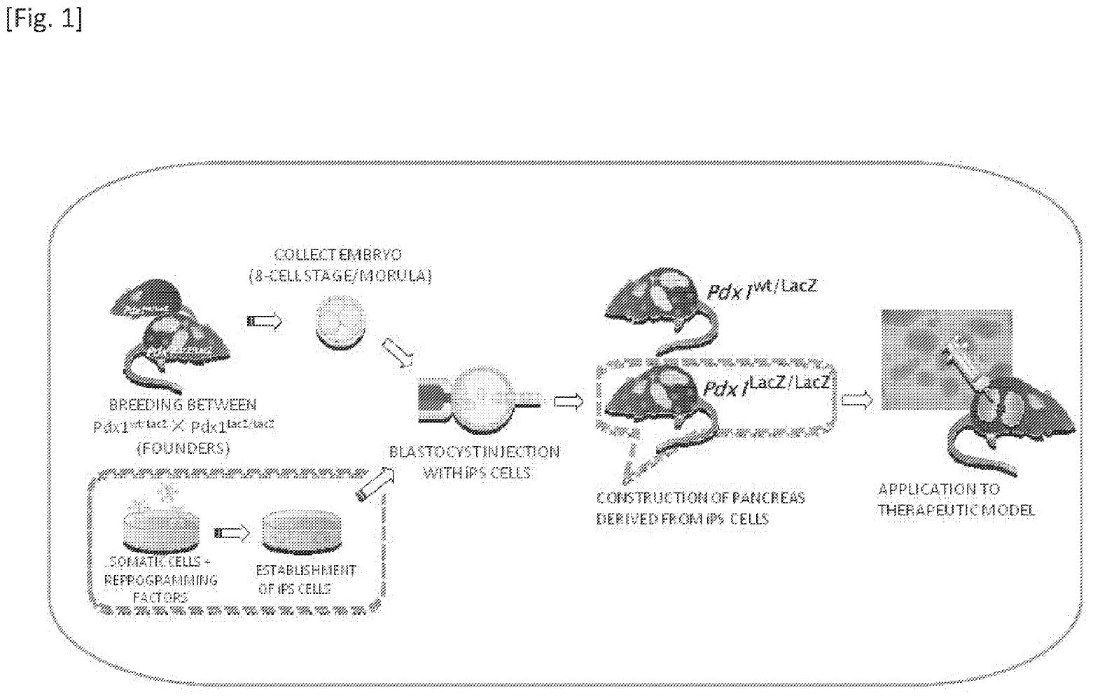

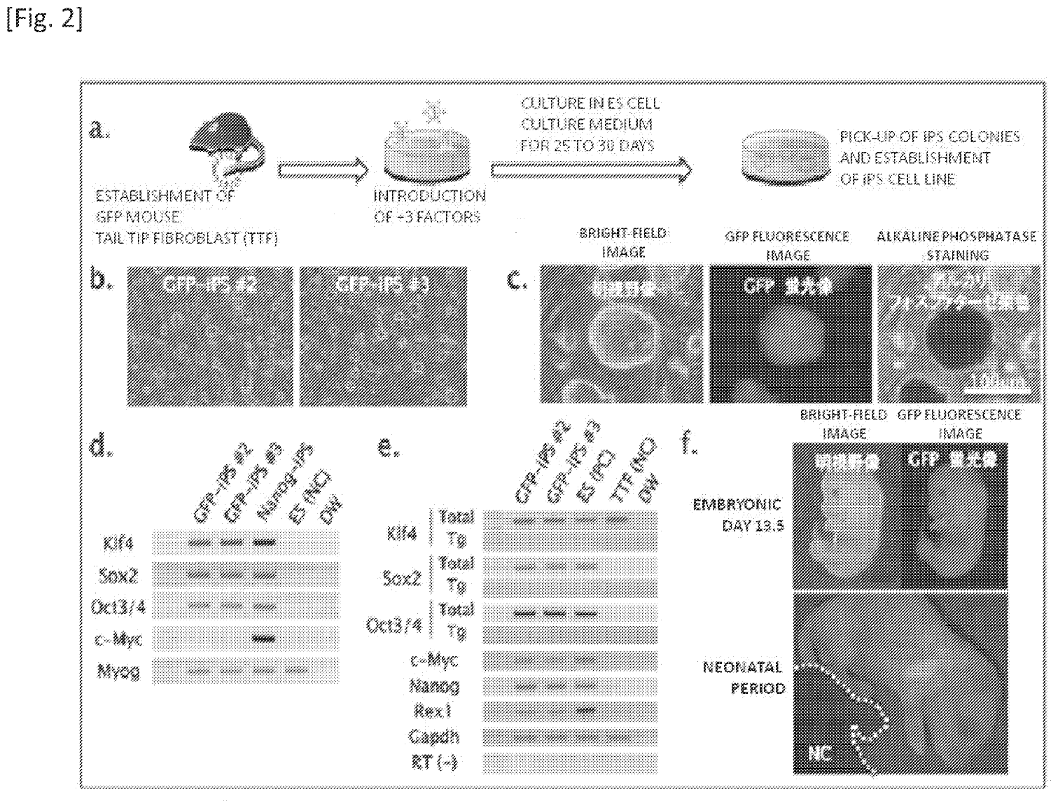

The inventors produced induced pluripotent stem (iPS) cells with 3 factors (Klf4, Sox2, and Oct3/4) by using a fibroblast collected from a tail of a GFP transgenic mouse. The protocol is as follows. The scheme is shown in FIG. 1, and shown in detail in FIG. 2a.

### (Establishment of GFP Mouse Tail Tip Fibroblast (TTF))

Approximately 1 cm of a tail of a GFP transgenic mouse was collected, peeled, and minced into 2 to 3 pieces. Then, these pieces were placed on MF-start medium (TOYOBO, Japan), and cultured for 5 days. Fibroblasts which appeared there were transferred to a fresh culture dish, and subcultured for several passages to be used as tail tip fibroblasts (TTF).

### (Introduction of +3 Factors (Reprogramming Factors))

A supernatant from a virus producing cell line (293gp or 293GPG cell line) produced by introducing a target gene and a virus envelope protein was collected, concentrated by centrifugation, and then frozen for preservation to be used as a virus fluid. The virus fluid was added to a culture fluid of TTF cells which had been subcultured on a previous day to achieve 1×10⁵ cells/6-well plate. This completed the introduction of the 3 factors (reprogramming factors).

### (Culture in ES-cell Medium for 25 to 30 Days)

On the next day after the introduction of the 3 factors (reprogramming factors), the culture fluid was replaced with a culture fluid for ES cell culture, and the culture was continued for 25 to 30 days. During this, culture fluid was replaced every day.

### (Pick-Up of iPS Colonies and Establishment of iPS Cell Line)

iPS cell-like colonies appeared after the culture were picked up using a yellow tip (for example, available from Watson), dissociated into single cells in 0.25% trypsin/EDTA (Invitrogen Corp.), and spread on a freshly prepared mouse embryonic fibroblast (MEF).

### (Result)

It was proved that the iPS cell line established by the above-described method had properties of iPS cells as shown in FIGS. 2b to f, that is, being undifferentiated and having totipotency.

FIG. 2 shows result of the above-described experiment. As shown in FIG. 2b, the morphology of two of thus established iPS cell lines was photographed by a microscope equipped with a camera. The conditions were as follows.

After subculturing the iPS cells after the pick-up, they were observed and photographed when they reached the semi-confluent stage on the dish.

It was found that morphologically ES cell-like undifferentiated colonies were formed.

As shown in FIG. 2c, the iPS cells were photographed under a fluorescent microscope, and subjected to staining using an alkaline phosphatase staining kit (Vector Laboratories, Inc., Cat. No. SK-5200). The conditions were as follows.

After observation and photographing of a bright-field image and a GFP fluorescence image by a microscope equipped with a camera, the culture fluid was removed from the iPS cell culture dish, and the dish was washed with a phosphate buffer saline (PBS). Then, a fixing solution containing 10% formalin and 90% methanol was added to the dish, thereby performing a fixing treatment for 1 to 2 minutes. After washing the resultant dish once with a washing solution (0.1 M Tris-HCl (pH 9.5)), a staining solution included in the above kit was added to the dish, and the dish was left to stand in dark for 15 minutes. Thereafter, the dish was again washed with the washing solution, and then observed and photographed.

As shown in FIG. 2c, it was found that, as having been derived from a GFP mouse, the iPS cells produced in the present example constantly expressed GFP, and showed a high level of alkaline phosphatase activity that is characteristic of undifferentiated cells.

As shown in FIG. 2d, for the purpose of identifying the 3 factors inserted into the genomic DNA in the establishment of the iPS cells, the genomic DNA was extracted from the iPS cells and subjected to PCR. The conditions were as described below.

The genomic DNA was extracted from 1×10⁶ cells using a DNA mini kit (Qiagen Co., Ltd.) according to the manufacturer's protocol. Using thus extracted DNA as a template, PCR was carried out using the primers below.

Fw (mOct3/4-S1120):
(SEQ ID NO: 1)
CCC TGG GGA TGC TGT GAG CCA AGG
Rv (pMX/L3205):
(SEQ ID NO: 2)
CCC TTT TTC TGG AGA CTA AAT AAA
Klf4
Fw (Klf4 -S1236):
(SEQ ID NO: 3)
GCG AAC TCA CAC AGG CGA GAA ACC
Rv (pMXs-AS3200):
(SEQ ID NO: 4)
TTA TCG TCG ACC ACT GTG CTG CTG
Sox2
Fw (Sox2-S768):
(SEQ ID NO: 5)
GGT TAC CTC TTC CTC CCA CTC CAG
Rv (pMX-AS3200):
(SEQ ID NO: 4)
same as above
c-Myc
FW (c-Myc-S1093):
(SEQ ID NO: 6)
CAG AGG AGG AAC GAG CTG AAG CGC
Rv (pMX-AS3200):
(SEQ ID NO: 4)
same as above

As a result, the insertion of the 3 factors was confirmed as shown in FIG. 2d.

As shown in FIG. 2e, a gene expression pattern unique to ES cell and expression of introduced genes were confirmed by reverse-transcription polymerase chain reaction (RT-PCR). The conditions were as described below.

1×10⁵ GFP-positive cells were sorted into Trizol-LS Reagent (Invitrogen Corp.) using a flow cytometer, mRNA was extracted from the cells, and cDNA was synthesized from the mRNA using ThermoScript RT-PCR System kit (Invitrogen Corp.) according to the attached protocol. Thus synthesized cDNA was used as a template to perform PCR. In regard to primers used, the same primers as those used in FIG. 2d above were used for transgene expression (notated as Tg in the drawing), while primers synthesized based on the report by Takahashi K & Yamanaka S (Cell 2006 Aug. 25; 126 (4): 652-5.) or the like were used for other gene expression.

As shown in FIG. 2e, all the lines showed expression patterns approximately same as that of ES cell. Further, it was found that expression of the introduced gene (Tg) was inhibited by a high level of gene silencing activity of the iPS cell.

As shown in FIG. 2f, thus established iPS cells were injected into a blastocyst to produce a chimeric mouse. The conditions were as follows.

Using an ovum collected from a BDF1 strain mouse (female, 8 weeks old) having been subjected to an ovarian hyperstimulation treatment by administration of PMSG and hCG hormones and a C57BL/6-derived sperm, in vitro fertilization (IVF) was performed to obtain a fertilized egg. The fertilized egg thus obtained was cultured to the 8 cell-stage/morula, then frozen for preservation, and recovered the day before blastocyst injection. In regard to the iPS cells, those reached the semi-confluent stage were detached using 0.25% Trypsin/EDTA, and suspended in ES-cell culture media to be used for injection. Blastocyst injection was performed, in the same manner as the technique used for the blastocyst complementation, under a microscope using a micromanipulator. Going through culture after the injection, transplantation into the womb of an ICR strain surrogate parent was performed. In analysis, observation and photographing were carried out under a fluorescence stereoscopic microscope on embryonic day 13 and postnatal day 1.

As shown in FIG. 2f, iPS cell-derived cells (GFP positive) were confirmed in the fetal period and the neonatal period. Accordingly, it was suggested that the established iPS cell line possessed high multipotency.

### Example 1

In the present example, a mouse was selected to be a founder animal, and pancreas was selected as an organ to be defected. Further, for preparation of a knockout mouse that was characterized by pancreas deficiency, a Pdx1 gene was used.

### (Mouse Used)

As a knockout mouse that was characterized by pancreas deficiency, Pdx1^{wt/LacZ} and Pdx1^{LacZ/LacZ} (founders) were used. A blastocyst derived from a mouse in which LacZ gene had been knocked in (also knocked out) at a Pdx1 gene locus (Pdx1-LacZ knock-in mouse) was used.

### (Pdx1-LacZ Knock-In Mouse)

In regard to the production of a construct, it can be produced based on specifically the published article (Development 122, 983-995 (1996)). In brief, the procedure is as follows. As for the arm of the homologous region, a product cloned from a λ clone including the Pdx1 region can be used. In the present example, an arm donated by Professor Yoshiya Kawaguchi at the Laboratory of Surgical Oncology, Kyoto University Graduate School of Medicine, was used.

### (Technique of Transgenic Knock-In: Pdx1-LacZ Knock-In Mouse)

A clone obtained by introducing the construct by electroporation into iPS cells prepared as described above, performing positive/negative selection, and then screening by Southern Blotting, was injected into a blastocyst so as to produce a chimeric mouse. Subsequently, a cell line developed into the germline is established, and the genetic background can be backcrossed into a C57BL/6 strain to produce the mouse.

### (Founder Mouse)

### (Mouse Used)

As a transgenic mouse characterized by pancreas deficiency, a mouse (Pdx1-Hes1 mouse) used was produced by injecting a construct in which a Hes1 gene is connected downstream of the Pdx1 promoter region into a mouse egg in the pronuclear stage. In regard to the production of the construct, the construct can be produced by inserting a Hes1 gene (an mRNA whose NCBI Accession Number is NM_008235) at the region of the Pax6 gene in a construct including the Pdx1 promoter region, the construct having been used in the published article (Diabetologia 43, 332-339 (2000).

### (Technique of Transgenic)

The above construct was injected using a microinjector into an egg in the pronuclear stage obtained from breeding between a C57BL6 mouse and a BDF1 mouse (purchased from Japan SLC, Inc.). A resulting egg was transplanted into a surrogate parent to produce a transgenic mouse.

The degree of formation of pancreas differs depending on the expression level of Hes1 which is expressed under the Pdx1 promoter (expressed especially in a fetal pancreas). When the expression is high (that is, the copy number is high), a deficiency of pancreas is indicated. Regeneration of pancreas by blastocyst complementation in the Pdx1-Hes1 transgenic mouse has been shown. Using this mouse, it is possible to produce a pancreas derived from iPS cells.

From this, it was demonstrated that the pancreas of a transgenic mouse thus produced could be complemented in the same manner as that of the Pdx1 knockout mouse.

### (Production of Founder Transgenic Mouse)

Since it has been known that the above-described transgenic mouse dies after birth, a founder of such a mouse is produced.

In a brief description, an embryo into which the Pdx1-Hes1 transgene is injected is cultured to become a blastocyst. An iPS cell or the like is injected into the thus obtained blastocyst under a microscope using a micromanipulator so as to complement a deficiency of pancreas. In this instance, an iPS cell marked with GFP or the like is used as the iPS cell as in the section of knockout. Alternatively, a marked iPS cell or the like which is equivalent to this may be used. The embryo after the injection is transplanted into the womb of a surrogate parent, and thus a litter can be obtained. When double embryo manipulations are applied in which a transgene is introduced into an embryo and then an iPS cell is injected into the embryo, it is possible to complement a pancreas in the first-generation transgenics. Accordingly, these may be a founder animal which is capable of transmitting the phenotype of pancreas deficiency to the next generation.

### (Breeding)

Next, in the present example, heterozygous mice of the mouse thus established were bred and used. In regard to the knock-in mice described above, since the mice could not survive homozygosity (died in approximately one week after birth), Pdx1^{wt/LacZ} and Pdx1^{LacZ/LacZ} (founders) were bred, and resulting embryos were recovered.

### (Procedure for Maintenance of Mouse and Confirmation)

The above-described iPS cells were injected into a blastocyst under a microscope using a micromanipulator (FIG. 1, Blastocyst injection with iPS cells). In a conventional method, it was necessary to mark the iPS cells with GFP. This time, however, since iPS cells were established from somatic cells of a GFP mouse in advance, they did not need to be marked, and used as they were. It is needless to say that other marked iPS cells or the like which are equivalent to this may also be used. The embryos after the injection were transplanted into the womb of a surrogate parent, and thus a litter was obtained.

For thus obtained litter, if the litter is knock-in mice, the probability of the animals being homozygous is ¼. For this reason, it is necessary to decide which mouse is the desired “pancreas-deficient+iPS cell-derived pancreas.” Therefore, a hit mouse was determined by collecting the cells of blood and tissues from both animals, isolating cells that were found to be GFP-negative (the cells were not derived from iPS cells, but derived from the injected embryos) by a flow cytometer, extracting the genomic DNA, and detecting the genotype by a PCR method. The primers used were as follows.

Forward (Fw):
(SEQ ID NO: 7)
ATT GAG ATG AGA ACC GGC ATG
Reverse 1 (Rv1):
(SEQ ID NO: 8)
TTC AAC ATC ACT GCC AGC TCC
Reverse (Rv2):
(SEQ ID NO: 9)
TGT GAG CGA GTA ACA ACC.

When produced by this method in which heterozygotes were bred, a resulting litter is expected to be wild type:heterozygote:KO=1:2:1 according to Mendelian inheritance. Accordingly, in order to specify a KO individual within the litter, genotyping was carried out using host-derived cells in peripheral blood as described above to specify the genotype.

As the first step to confirm whether or not the complemented organ functioned normally, analysis of expression of functional markers was carried out in the neonatal period in which morphological observation of pancreas was easy.

Images of frozen sections which were prepared from pancreases of mice dissected in the neonatal period and then subjected to immunostaining are shown. Images of those which were stained using an anti-insulin antibody (purchased from NICHIREI Biosciences Inc., cat. #422421) as a marker of endocrine tissue as an antibody are shown. In addition to this, staining may be carried out using each of: an anti-a-amylase antibody (purchased from SIGMA CORPORATION, cat. #A8273), an anti-glucagon antibody (purchased from NICHIREI Biosciences Inc., cat. #422271), and an anti-somatostatin antibody (purchased from NICHIREI Biosciences Inc., cat. #422651) as markers of exocrine tissue; and a DBA-Lectin (purchased from Vector Laboratories, cat. #RL-1032) as a marker of pancreatic duct. As being positive to insulin, it was understood without carrying out staining with other antibodies that the complemented pancreas functioned normally.

From this result, expression of almost all the functional markers was confirmed. Therefore, it was inferred that the complemented pancreas had normal functions which were enough for its survival.

Then, as the next step to confirm whether or not the complemented organ functioned normally, measurement of blood glucose level was carried out on adult mice.

Result of pancreatic function evaluation using blood glucose level as an indicator carried out on mice having a pancreas complemented can be taken into consideration. Averages and standard deviations of steady state blood glucose levels, which were measured using Medisafe Mini GR-102 (purchased from TERUMO CORPORATION), of matured mice having a pancreas complemented may be taken into consideration. As controls, levels of chimeric mice having Pdx1 alleles in a heterozygous state and an STZ-DM model having a decreased pancreatic function may be used. Further, result of measurement of changes in the blood glucose level after a glucose tolerance test may be taken into consideration, the measurement being carried out using the above-described Medisafe Mini. These results showed normality of the ability to regulate blood glucose level, and indicated that the thus produced mice having a pancreas complemented were able to survive over a long period of time even when used as a founder.

Specifically, as having a blood glucose level, which was once elevated but had gone back to the normal level after the glucose tolerance test similarly to that of the hetero (+/−) chimera used as a control, the produced KO chimeras (founders) did not show any symptoms of diabetes and the like. Thus, the possibility of long-term survival can be indicated.

Next, it was attempted to reveal, using a litter obtained from breeding with a hetero individual, whether or not the KO chimera can transmit the phenotype to the next generation as a founder mouse. Genomic DNA extracted from a tail of a thus obtained litter was subjected to PCR using the primers used in the section (Procedure for Maintenance of Mouse and Confirmation). As a result, it was revealed that only hetero or knockout individuals could be obtained. This strongly suggested that the founder was a knockout individual and capable of transmitting the phenotype to the next generation.

Specifically, since the breeding was performed between knockout (KO) and hetero mice, an obtained litter was expected to be a KO or hetero individual theoretically at a probability of ½ according to Mendelian inheritance. Then, result as expected was demonstrated.

This allows obtaining of a KO individual at a probability of 100% in the next generation in breeding between KO individuals each having, for example, a pancreas complemented. Accordingly, it is expected that analysis using a KO individual will be able to be carried out much more easily.

### (Confirmation of Chimera)

Chimeras can be determined by their hair colors. Since the donor iPS cells were derived from the GFP transgenic mouse and the host embryo was derived from C57BL6xBDF1 (black), which is a wild type, it was possible to determine according to GFP fluorescence. Determination of transgenic was carried out by detecting the transgene by PCR on genomic DNA extracted from a tail thereof.

If the litter is transgenic, the probability of the transgene being transmitted to the next generation is ½. For this reason, it is necessary to decide which mouse has the desired “pancreas-deficient+iPS cell-derived pancreas.” Therefore, individuals of the litter were each bred with a wild type mouse. Then, transmission of the transgene was confirmed by detecting a genotype by PCR on genomic DNA extracted from tails of a resulting litter, and the morphology of pancreases of the litter was observed. The following primer set was used for the PCR.

Forward (Fw):
(SEQ ID NO: 10)
TGA CTT TCT GTG CTC AGA GG
Reverse (Rv):
(SEQ ID NO: 11)
CAA TGA TGG CTC CAG GGT AA

The forward primer used was prepared so as to hybridize with a nucleotide sequence corresponding to the Pdx1 promoter region, while the reverse primer was prepared so as to hybridize with a nucleotide sequence of Hes1 cDNA (an mRNA whose Accession Number is NM_008235). Since such a Pdx1 promoter and Hes1 cDNA existing in the neighborhood cannot occur in wild type mice, it is possible to detect a transgene efficiently by PCR using these primers.

From the experiment above, result was obtained which suggested that the thus obtained litter individual was a founder mouse capable of causing pancreas deficiency in the next generation.

It is expected that application of such a method not only to mice but other large-size animals and the like will allow more efficient production of transgenic animals and knockout animals having a lethal phenotype.

The thus produced transgenic and chimeric individual was bred with a wild type. It was to be revealed whether or not the phenotype of pancreas deficiency was transmitted to the next generation by carrying out morphological analysis of pancreases of a litter or PCR on genomic DNA. If a transgenic-chimera can be a founder, a mouse having pancreas deficiency should be born in the next generation. One successful in causing deficiency of pancreas in the next generation can be selected. It is suggested that such a mouse showed normality after birth as its pancreas was complemented during the production. Even when a transgenic is used as described above, it is made possible to achieve organ regeneration with iPS cells by using a founder allowing efficient production of mice, such as an organ deficient mouse, which would die in the neonatal period and immediately after birth.

From the above, it was demonstrated that organ deficient animals, even mice including ones with pancreatic agenesis due to forced expression of HES-1, and even Pdx1 knockout mice, can be rescued from death using iPS cells by blastocyst complementation and used as founders.

### (Regeneration of Pancreas)

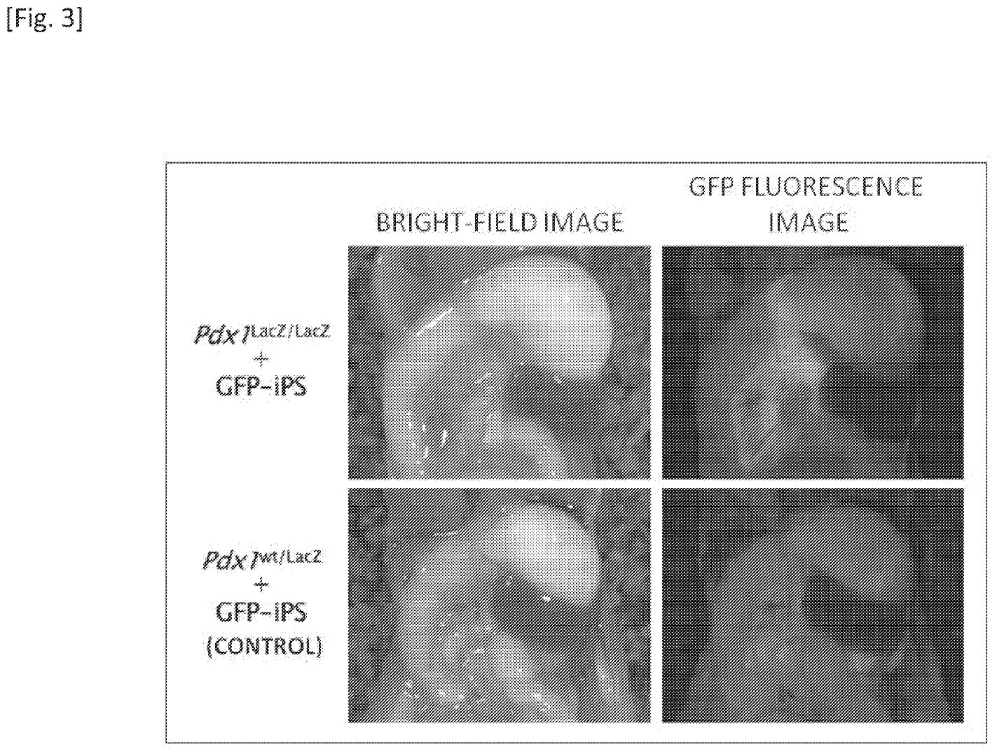

FIG. 3 shows regeneration of pancreases. Here, neonates 5 days after birth were dissected under a microscope, and pancreases were exposed. The thus obtained pancreases were observed and photographed under a fluorescent microscope. FIG. 3 shows the resulting photographs.

(Morphologies of Pancreases Derived from iPS Cells)

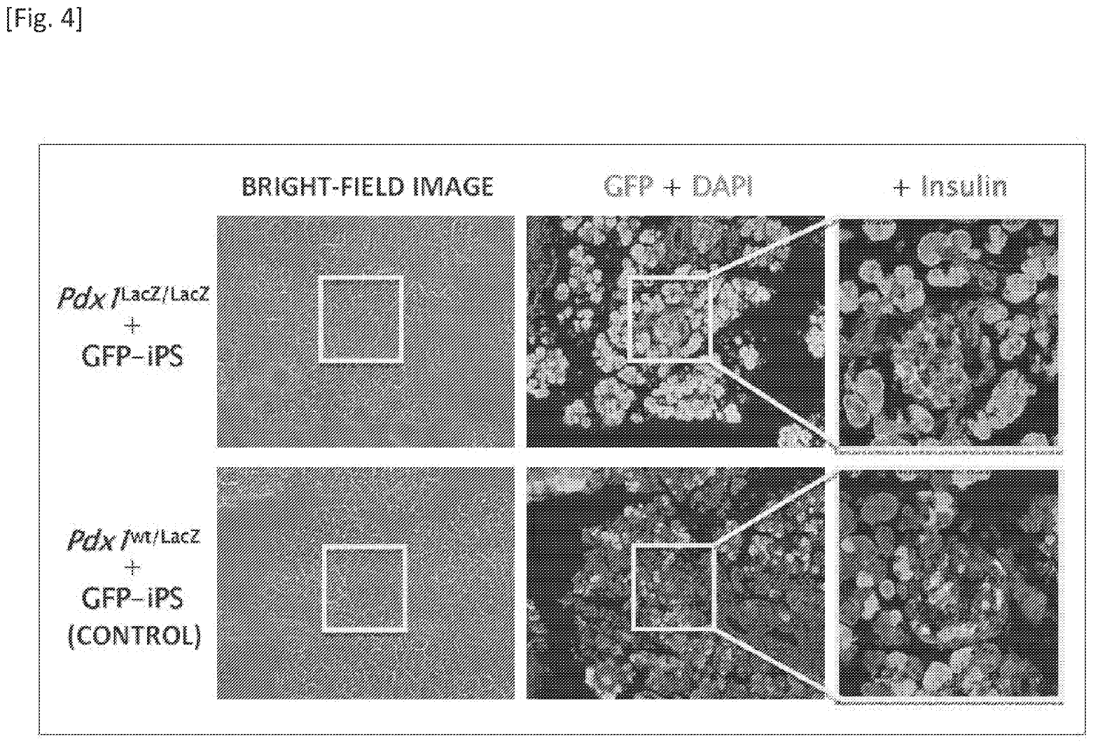

FIG. 4 shows the morphologies of pancreases derived from iPS cells. Here, frozen section samples of pancreases derived from iPS cells were prepared, subjected to nuclear staining with DAPI and an anti-GFP antibody and with and an anti-insulin antibody, and then observed and photographed using an upright fluorescent microscope and a confocal laser microscope.

From FIGS. 3 and 4, it is inferred that blastocyst complementation was accomplished morphologically.

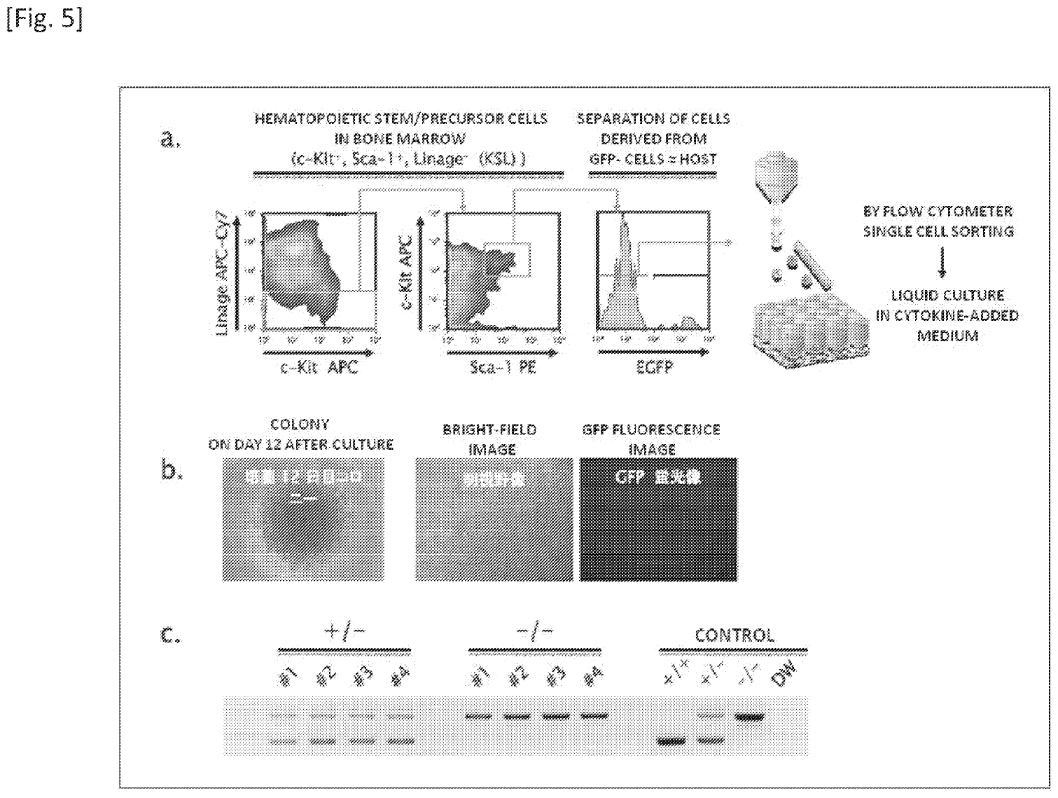

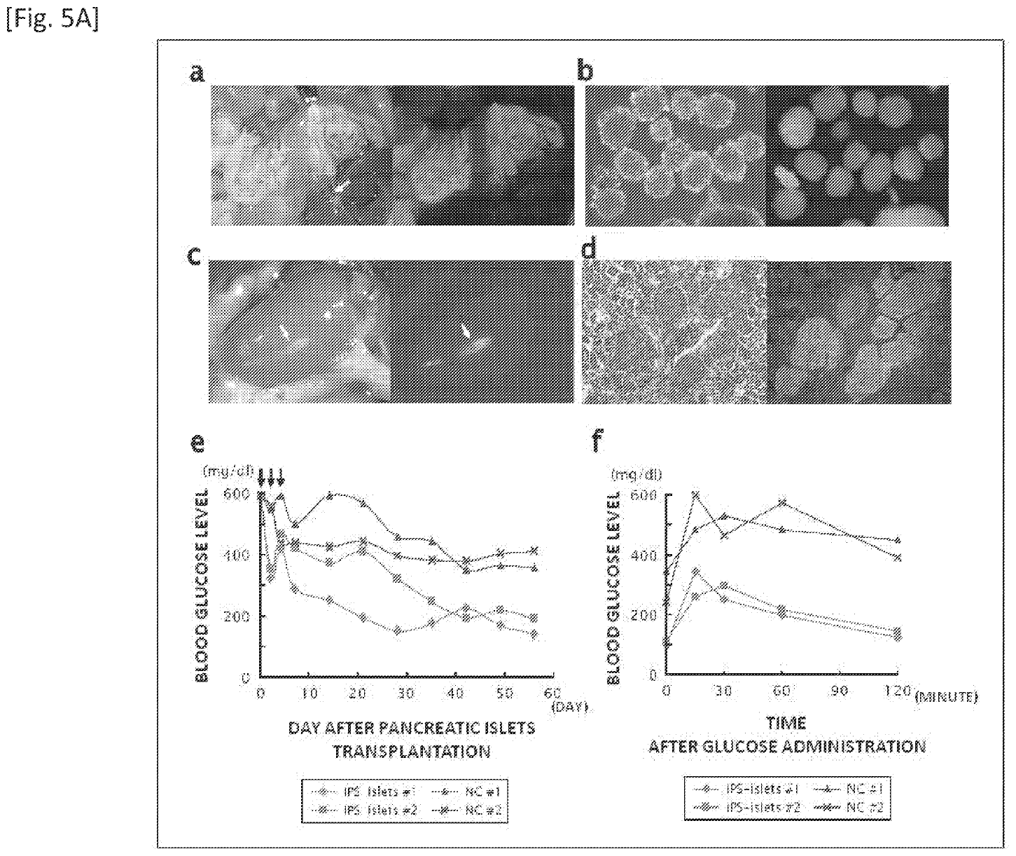

FIG. 5 shows a method for genotyping the host mouse. Bone marrow cells were collected from the mouse shown in FIG. 3, isolating hematopoietic stem/precursor cells (c-Kit+, Sca-1+, Linage marker-: KSL cells) that were found to be GFP-negative by a flow cytometer, and thus isolated cells were dropped onto a 96-well plate one by one. The cells were cultured under the condition of cytokine addition for 12 days to allow formation of colonies. Genomic DNA was extracted from these colonies, and used for genotyping. This enables clonal genotyping on a single cell even if cells whose GFP expression is blocked by the gene silencing are included on the GFP− side. A host cell and a cell subjected to gene silencing can be conveniently discriminated. Note that, in the experiment of FIG. 5, as an experiment for confirming the basis of establishment of blastocyst complementation (the basis is vacancy of an organ (i.e., knockout (KO)), and what was confirmed was this), and to confirm KO by genotyping for sure, genotyping was carried out on a single cell while the influence of gene silencing was being taken into consideration (FIG. 5). a. shows the strategy. b. shows images of the colonies formed after the culture. c. shows the determination result.

### (Discussion)

As described above, the organ regeneration was demonstrated with iPS cells which were uniquely produced using 3 factors (Klf4, Sox2, Oct3/4) and a fibroblast collected from a tail of a GFP transgenic mouse. Since homozygous and heterozygous Pdx1 knockout mice were bred, a homozygous pancreas deficiency mouse was expected to be born at a probability of 50%. This was demonstrated to be true. Moreover, from the morphology of the pancreas derived from iPS cells shown in FIG. 4 and from the result of PCR performed after separation and collection of GFP-positive and GFP-negative cells as shown in FIG. 5, a homozygous pancreas deficiency mouse was expected to be born at a probability of 50%. This was demonstrated to be true.

(Transplantation of iPS-Derived Pancreatic Islets into STZ-induced Diabetic Mice)

### (Mice Used)

A C57BL/6 mouse, a BDF1 mouse, a DBA2 mouse and an ICR mouse were used, which were purchased from Japan SLC, Inc. A Pdx1 heterozygous (Pdx1 (+/−)) mouse (donated by Professor Yoshiya Kawaguchi of Kyoto University Graduate School of Medicine and Dr. Wright of Vanderbilt University) was bred with the DBA2 mouse or BDF1 mouse. The C57BL/6 mouse was used as a donor of a streptozotocin (STZ)-induced diabetic model. After fasting for 16 to 20 hours, STZ (200 mg/kg) was intravenously administered. A mouse with a blood glucose level exceeding 400 mg/dL one week after this STZ injection was considered as a high blood sugar diabetes mouse.

(Culture of mES/miPS Cell)

Undifferentiated mouse embryonic stem (mES) cells (G4.2) were placed on a gelatin-coated dish and maintained in a Glasgow's modified Eagle's medium (GMEM; Sigma Corporation, St. Louis, MO) without feeder cells, the GMEM being supplemented with 10% fetal bovine serum (FBS; from NICHIREI Biosciences Inc.), 0.1 mM 2-mercaptoethanol (Invitrogen Corp., San Diego, CA), 0.1 mM non-essential amino acid (Invitrogen Corp.), 1 mM sodium pyruvate (Invitrogen Corp.), 1% L-glutamine penicillin streptomycin (Sigma Corporation), and 1000 U/ml leukemia inhibitory factor (LIF; Millipore, Bedford, MA). The G4.2 cells (which were donated by Professor Niwa Hitoshi at RIKEN CDB) are derived from an EB3 ES cell, and carry an enhanced green fluorescence protein (EGFP) gene under the control of the CAG expression unit. The EB3 ES cell is a subline cell derived from E14tg2a ES cells (Hooper M. et al., 1987), and established by targeting, on Oct-3/4 allele, the incorporation of an Oct-3/4-IRES-BSD-pA vector constructed so as to express blasticidin, which is a drug-resistance gene, under the control of Oct-3/4 promoter (Niwa H. et al., 2000).

Undifferentiated mouse induced pluripotent stem (miPS) cells (GT3.2) were maintained on a mitomycin C-treated mouse embryonic fibroblast (MEF) in Dulbecco's modified Eagle's medium (DMEM; Invitrogen Corp.) supplemented with 15% knockout serum replacement (KSR; Invitrogen Corp.), 0.1 mM 2-mercaptoethanol (Invitrogen Corp.), 0.1 mM non-essential amino acid (Invitrogen Corp.) 1 mM HEPES buffer solution (Invitrogen Corp.), 1% L-glutamine penicillin streptomycin (Sigma Corporation), and 1000 U/ml leukemia inhibitory factor (LIF; Millipore) The GT3.2 cells were established from a fibroblast collected from a tail of a male GFP transgenic mouse (donated by Professor Okabe Masaru at Osaka University) into which 3 reprogramming factors, Klf4, Sox2, Oct3/4, were introduced with a retrovirus vector. The GT3.2 cells ubiquitously express EGFP under the control of the CAG expression unit.

### (Culture and Manipulation of Embryo)

An embryo resulting from outcross of Pdx1 heterozygotes (Pdx1 (+/−)) was prepared according to the published protocol (Nagy A. et al., 2003). In a brief description, mouse 8-cell/morula embryos were collected from the oviduct and womb on day 2.5 after outcrossing of the Pdx1 heterozygous mice into M2 medium (Millipore). These embryos were transferred into drops of KSOM-AA medium (Millipore), and cultured to the blastocyst stage for 24 hours.

For embryo manipulation, the blastocyst was transferred into fine drops containing M2 medium. The mES/miPS cells were treated with trypsin, and then suspended in the fine drops of the culture medium. At the 8-cell/morula stage, the embryos were transferred into the fine drops containing HEPES buffer mES/miPS culture medium. Using a piezo-actuated micromanipulator (manufactured by Primetech Corporation), pores were carefully created in the zona pellucida and trophectoderm under a microscope. Then, 10 to 15 mES/miPS cells were injected near the inner cell mass (ICM) in the blastocyst cavity. After the injection, the embryos were cultured in KSOM-AA medium for 1 to 2 hours, and thereafter transplanted into the womb of a surrogate parent female ICR mouse on 2.5 dpc bred for pseudo-pregnancy.

### (Isolation and Transplantation of Pancreatic Islets)

By collagenase digestion, pancreatic islets were isolated from a mouse having an iPS-derived pancreas, and separated into pieces by ficoll gradient centrifugation. In a brief description, 10- to 12-week old adult mouse was sacrificed, and using a 27-G butterfly needle, the pancreas was perfused via the bile duct with 2 mg/ml collagenase (manufactured by YAKULT HONSHA CO., LTD.) in Hanks' balanced salt solution (HBSS: Invitrogen Corp.). The perfused pancreas was dissected and incubated at 37° C. for 20 minutes. The thus digested fractions were washed twice with HBSS, and undigested tissues were removed using a strainer. The resulting fractions were separated by density-gradient centrifugation using Ficoll PM400 (GE-Healthcare, Stockholm, Sweden) in HBSS, and the fractions of concentrated pancreatic islets were collected into RPMI medium (Invitrogen Corp.) containing 10% FCS. The pancreatic islets having a diameter exceeding approximately 150 μm were collected using a glass micropipette into a tube under a microscope.

Using a glass micropipette, 150 pancreatic islets thus isolated were transplanted into the STZ-induced diabetic mice through the kidney films. In order to prevent the thus transplanted pancreatic islets from disappearing immediately, a reported anti-inflammatory monoclonal antibody cocktail [containing anti-mouse IFN-γmonoclonal antibody (mAb) (R4-6A2; rat IgGκ:e-Bioscience), anti-mouse TNF-α mAb (MP6-XT3; rat IgG1κ:e-Bioscience), and anti-mouse IL-1β mAb (B122; American hamster IgG:e-Bioscience)] was administered into the intraperitoneal cavity three times on day 0, day 2, and day 4 after the transplantation.

### (Immunohistochemistry)

Two months after the transplantation of pancreatic islets, observation of GFP expression (indicating the transplanted pancreatic islets) was carried out. HE staining and GFP staining with DAPI were performed on a kidney section, and the presence of the transplanted pancreatic islets was confirmed.

### (Monitoring of Blood Glucose Level)

The blood glucose level of mice which were not subjected to fasting was monitored by collecting blood samples when the mAb was administered and every one week for two months after the transplantation of the pancreatic islets. The blood glucose level was measured using Medisafe Mini GP-102 (purchased from TERUMO CORPORATION). Moreover, a glucose tolerance test (GTT) was performed two months after the transplantation of the pancreatic islets.

The data are shown in FIG. 5A. FIG. 5A shows transplantation of iPS-derived pancreatic islets into an STZ-induced diabetic mouse. a and b show the isolation of the pancreatic islets. The iPS-derived pancreas was perfused via the common bile duct (arrow in a.) with collagenase. After density-gradient centrifugation, iPS-derived pancreatic islets that expressed EGFP were concentrated (b). c shows the kidney film two months after the transplantation of the pancreatic islets. A spot (arrow) where EGFP was expressed is the transplanted pancreatic islet. d shows HE staining (left panel) and GFP staining with DAPI (right panel) performed on the kidney section. e shows the transplantation of 150 iPS-derived pancreatic islets into the STZ-induced diabetic mice. Arrows indicates the time when the antibody cocktail (anti-INF-γ, anti-TNF-α, anti-IL-1β) was administered. The blood glucose level in the intraperitoneal cavity was measured every one week until two months elapsed after the transplantation. The STZ-induced diabetic mice into which the iPS-pancreatic islets were transplanted were represented by ▴ (black triangles) (n=6), while the STZ-induced diabetic mice into which no iPS-pancreatic islets were transplanted were represented by ▪ (black squares). f shows the glucose tolerance test (GTT) performed two months after the transplantation of the pancreatic islets.

As described above, it was shown from the result in FIG. 5A that the symptom of diabetes was improved by transplantation of iPS-derived pancreatic islets. This indicates the therapeutic effect of the organ regeneration technique using iPS.

### Example 2: Example in Case of Kidney

In accordance with Example 1, organ regeneration of kidney was performed.

In the present example, it was investigated whether or not kidney development would occur by transplanting, as pluripotent cells, mouse iPS cells produced as described above into a knockout mouse that was characterized by kidney deficiency.

As the knockout mouse characterized by kidney deficiency, a Sall1 knockout mouse (donated by Professor Ryuichi Nishinakamura at Institute of Molecular Embryology and Genetics, Kumamoto University) was used. Sall1 gene is a gene of 3969 bp, encoding a protein having 1323 amino acid residues. This gene is a mouse homolog of the anterior-posterior region-specific homeotic gene spalt (sal) of Drosophila, and has been suggested by a pronephric tubule induction test in African clawed frogs to be important in kidney development (Nishinakamura, R. et al., Development, Vol. 128, p. 3105-3115, 2001, Asashima Lab, Tokyo University). It was reported that this Sall1 gene was expressed and localized in the kidney, as well as in the central nervous system, auditory vesicles, heart, limb buds and anus in the mouse (Nishinakamura, R. et. al., Development, Vol. 128, p. 3105-3115, 2001).

The knockout mouse having this Sall1 gene (backcrossed to C57BL/6 strain and analyzed) has exon 2 and its subsequent parts in the Sall1 gene deleted, and thereby lacking all of the 10 zinc finger domains present in the molecule. It is conceivable that, as a result of the deletion, interpolation of ureteric bud into the metanephric mesenchyme does not occur, thereby causing abnormality in the initial stage of kidney formation (normal individual, Sall1 knockout mouse).

The genotyping of the Sall1 knockout mouse used in the experiment was carried out in the same manner as the method for genotyping the host mouse shown in FIG. 5. Bone marrow cells were collected from the mouse, isolating hematopoietic stem/precursor cells (c-Kit+, Sca-1+, Linage marker-: KSL cells) that were found to be GFP negative by a flow cytometer, and thus isolated cells were dropped onto a 96-well plate one by one. The cells were cultured under the condition of cytokine addition for 12 days to allow formation of colonies. Genomic DNA was extracted from these colonies, and used for genotyping. Note that, as an experiment for confirming the basis of establishment of blastocyst complementation (the basis is vacancy of an organ (i.e., knockout (KO)), and what was confirmed was this), and to confirm KO by genotyping for sure, genotyping was carried out on a single cell.

The primers used for the genotyping were as follows. Forward primer for identification of one derived from injected embryo (that is, a host):

For detection of mutant:
(SEQ ID NO: 12)
AAG GGA CTG GCT GCT ATT GG
For detection of wild type:
(SEQ ID NO: 13)
GTA CAC GTT TCT CCT CAG GAC
Reverse primer for identification of one derived
from injected embryo (that is, a host):
For detection of mutant:
(SEQ ID NO: 14)
ATA TCA CGG GAT GCC AAC GC
For detection of wild type:
(SEQ ID NO: 15)
TCT CCA GTG TGA GTT CTC TCG

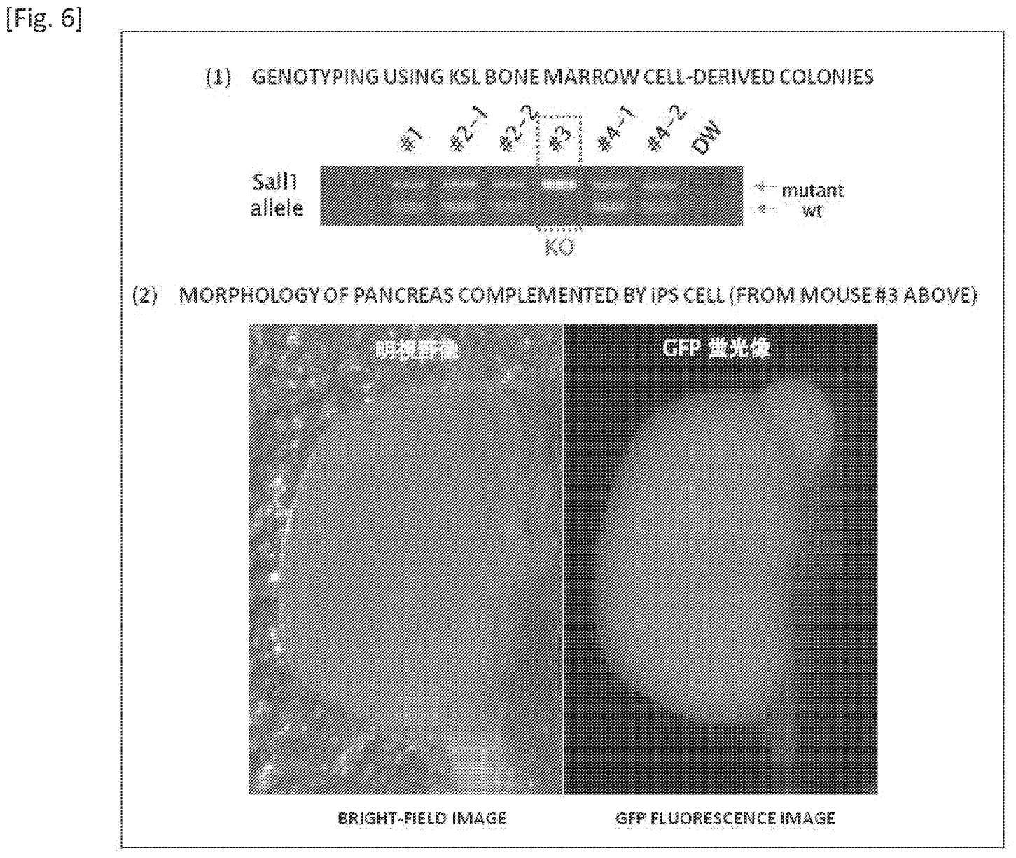

When produced by this method in which heterozygotes were bred, a resulting litter is expected to be wild type:heterozygote:KO=1:2:1 according to Mendelian inheritance. Accordingly, in order to specify a KO individual within the litter, genotyping was carried out using bone marrow cells as described above to specify the genotype (FIG. 6). It was found that mouse #3 was a Sall1 homo KO mouse.

By performing such genotyping, it is possible to confirm that genotyping on a chimeric individual is possible.

An investigation was made on the kidney formation in the individuals of a mouse litter one day after birth, which have been found to be homozygotes (Sall1 (−/−)) or heterozygotes (Sall1 (+/−)) according to the genotyping. It can be understood that kidneys were formed in the heterozygotes (Sall1 (+/−)), but kidneys were not at all formed in the homozygotes (Sall1 (−/−))

Next, male and female heterozygous individuals (Sall1 (+/−)) of the Sall1 gene knockout mouse were bred, and thus the blastocyst stage fertilized eggs were collected by a uterine reflux method. The genotype of the blastocyst stage fertilized eggs obtained as described above was expected to appear at a ratio of homozygote (Sall1 (−/−)):heterozygote (Sall1 (+/−)):wild type (Sall1 (+/+))=1:2:1.

The GFP-marked iPS cells described above were injected by microinjection into the collected blastocyst stage fertilized eggs with 15 cells per blastocyst. The eggs were returned to the womb of a surrogate parent (ICR mouse, purchased from Japan SLC, Inc.).

The neonatal chimeric individuals, which could be confirmed to be homozygotes (Sall1 (−/−)) by the above-described genotyping, were confirmed to have kidneys present in the retroperitoneal area. When these formed kidneys were observed under a fluorescent stereoscopic microscope, GFP-positive findings were confirmed (FIG. 6). This indicates that, in the homozygotes (Sall1 (−/−)), the kidneys were derived only from the mouse iPS cells transplanted into the inner space of the blastocyst stage fertilized eggs. On the other hand, in the heterozygous (sall1 (+/−)) individuals, since the kidneys were constituted of a chimera of the cells, which were derived from the heterozygous (Sall1 (+/−)) individuals, and the cells, which were derived from the transplanted iPS cells, confirmation was performed by obtaining cellular images that were positive for both the GFP fluorescence and the immunohistochemically derived fluorescence using an anti-GFP antibody.

In the histological analysis of the kidneys obtained as a result of transplanting iPS cells into the homozygous (Sall1 (−/−)) blastocyst stage fertilized eggs, mature functional glomeruli, which contained erythrocytes, in the loop cavity and mature renal tubular structures were observed. Those mature cells could be confirmed to be mostly GFP-positive by an immunohistochemical analysis using an anti-GFP antibody.

From the above, it can be confirmed that, in the chimeric Sall1 knockout mouse (Sall1 (−/−)) created by the method described above, the kidney formed in the litter individual was formed from the iPS cell that had been transplanted into the inner space of the blastocyst stage fertilized egg of the Sall1 knockout mouse (Sall1 (−/−)).

### Example 3: Hair Development in Hair-Deficient Mouse Strain

In regard to hair, it was investigated whether or not hair development would occur by using nude mouse-derived blastocysts, and transplanting, as pluripotent stem cells, mouse iPS cells produced above.

### (Mouse Used)

The mouse used was a nude mouse purchased from Japan SLC, Inc. The nude mouse used was a sturdy nude mouse having a good breeding efficiency, which was produced when nu gene of a BALB/c nude was introduced into an inbred DDD/1 strain mouse.

Mouse iPS cells were injected into blastocysts under a microscope using a micromanipulator. Mouse iPS cells into which GFP was introduced were used as the mouse iPS cells. Alternatively, a marked mouse iPS cell or the like which is equivalent to this may be used. The embryo after the injection was transplanted into the womb of a surrogate parent, and a litter was obtained.

The nude mouse is a spontaneous model. The mouse is deficient of thymus and hair, but does not cause any impediment in the survival and propagation. Accordingly, breeding between nude mice is possible. Thus, all the litter individuals become nude mice, and genotyping is not necessary. Therefore, the confirmation by detection with PCR as in the case of Examples above is also unnecessary.

Whether a hair was developed or not was confirmed with the naked eye. This is a real example where a nude mouse developed hair by the method of the present invention. From this result, what was developed was a GFP-positive hair, it was verified that a hair could be regenerated even using a mouse iPS cell.

### (Conclusion)

From the above, it was found that a hair could be regenerated even with a mouse iPS cell using the method of the present invention.

### Example 4: Thymus Development in Thymus-Deficient Mouse Strain

In regard to thymus, it was investigated whether or not thymus development would occur by using nude mouse-derived from blastocysts, and transplanting, as pluripotent cells, mouse iPS cells produced as described above.

### (Mouse Used)

The mouse used was a nude mouse purchased from Japan SLC, Inc. The nude mouse used was a sturdy nude mouse having a good breeding efficiency, which was produced when nu gene of a BALB/c nude mouse was introduced into an inbred DDD/l strain mouse.

### (Procedure for Maintenance of Mouse and Confirmation)

Mouse iPS cells were injected into blastocysts under a microscope using a micromanipulator. The mouse iPS cells had GFP introduced therein. Alternatively, a marked mouse iPS cell or the like which is equivalent to this may be used. The embryo after the injection was transplanted into the womb of a surrogate parent, and a litter was obtained. In the present example, since the nude mouse was used, confirmation by PCR is not necessary as described in Example 3.

To see whether a thymus was developed or not, staining with CD4-positive and CD8-positive T cells was performed. This shows that if a thymus is present, the differentiation of matured T cells is induced, whereas if a thymus is not regenerated, the differentiation of mature T cells is not induced, indicating no thymus is present. Nonetheless, when normal iPS cells marked with GFP were introduced into the blastocyst of the nude mouse (BC, blastocyst complementation), both of the GFP-negative T cells (derived from of hematopoietic stem cells of the host nude mouse) and the GFP-positive T cells (derived from the iPS cells) were induced to differentiate. Thus, it was confirmed even from a functional viewpoint that a thymus was established by the mouse iPS cells.

Furthermore, to see the development of thymus in a nude mouse, a wild type mouse, and a chimeric mouse of the present invention, photographs were taken for confirmations. The photographs includes: photographs of the thymus of the wild type mouse, one showing the normal state and the other showing the fluorescence-illumination state; photographs of the thymus of the nude mouse, one showing the normal state and the other showing the fluorescence-illumination state; photographs of the thymus of the chimeric mouse produced through blastocyst complementation as described above, one showing the normal state and the other showing the fluorescence-illumination state; and a photograph of the thymus extracted from this chimeric mouse and illuminated with fluorescence. By the confirmation of the thymus exhibiting fluorescence, it was proved that a tissue was derived from the mouse iPS cells.

### (Conclusion)

From the above, it was found that a thymus could be regenerated even with a mouse iPS cell using the method of the present invention.

### Example 5

In the present example, xenogeneic blastocyst complementation was investigated using a Pdx1 knockout mouse that was characterized by pancreas deficiency as a host animal and using, as a donor cell, rat iPS cells (EGFP+) produced in accordance with the above preparation example.

### A. Animals Used

As a knockout mouse that was characterized by pancreas deficiency, a heterozygous individual (Pdx1 (+/−)) of a Pdx1 gene knockout mouse and a homozygous individual (Pdx1 (−/−): founder) having a pancreas complemented by a mouse iPS cell were used as in the case of Example 1.

### B. Preparation of Rat iPS Cells

### 1) Construction of Vector for Preparation of Rat iPS Cells

TRE from pTRE-Tight (Clontech), ubiquitin C promoter, tTA from pTet-on advanced (Clontech), and IRES2EGFP from pIRES2EGFP (Clontech) were incorporated into lentivirus vector CS-CDF-CG-PRE multicloning sites from 5′ end. Mouse Oct4, Klf4, and Sox2 were ligated to each other with F2A and T2A derived from a virus, and inserted between the TRE and the ubiquitin C promoter of the lentivirus vector for the production (LV-TRE-mOKS-Ubc-tTA-I2G).

### 2) Establishment of Rat iPS Cells

Wistar rat embryonic fibroblast (E14.5) cells, which were subcultured within 5 passages, were spread on a dish coated with 0.1% gelatin, and cultured in DMEM with 15% FCS and 1% penicillin/streptomycin/L-glutamine (SIGMA CORPORATION). On the following day of the inoculation, a lentivirus produced using the LV-TRE-mOKS-Ubc-tTA-I2G vector was added to the culture fluid to infect the cells with the virus. Twenty four hours later, the medium was replaced. The resultant cells were placed on MEF treated with mitomycin C, and cultured in DMEM containing 1 μg/ml doxycycline and 1000 U/ml rat LIF (Millipore) supplemented with 15% FCS and 1% penicillin/streptomycin/L-glutamine. From the following day, the medium was replaced with a serum-free N2B27 medium (GIBCO) supplemented with 1 μg/ml doxycycline and 1000 U/ml rat LIF (Millipore) every other day. From day 7, inhibitors (2i;3 mM CHIR99021 (Axon), 1mMPD0325901 (Stemgent), 3i;2i+2 mM SU5402 (CalbioChem)) were added. Colonies having appeared on day 10 and later were picked up, and transplanted onto a MEF feeder. Thus established riPS cells were maintained by subculturing using trypsin-EDTA every 3 to 4 days, and introduced into a blastocyst of a non-human mammal.

### C. Xenogeneic Blastocyst Complementation

A male Pdx1 (−/−) mouse was bred with a female Pdx1 (+/−) mouse, and the fertilized eggs were collected by a uterine reflux method. The fertilized eggs thus collected were developed to the blastocyst stage in vitro. The above rat iPS cells marked with EGFP were injected under a microscope by microinjection into the resultant blastocysts with 10 cells per blastocyst. The blastocyst was transplanted into the womb of a pseudo-pregnant surrogate parent (ICR mouse, purchased from Japan SLC, Inc.). Laparotomy was performed in the full term pregnancy, and neonates thus born were analyzed.

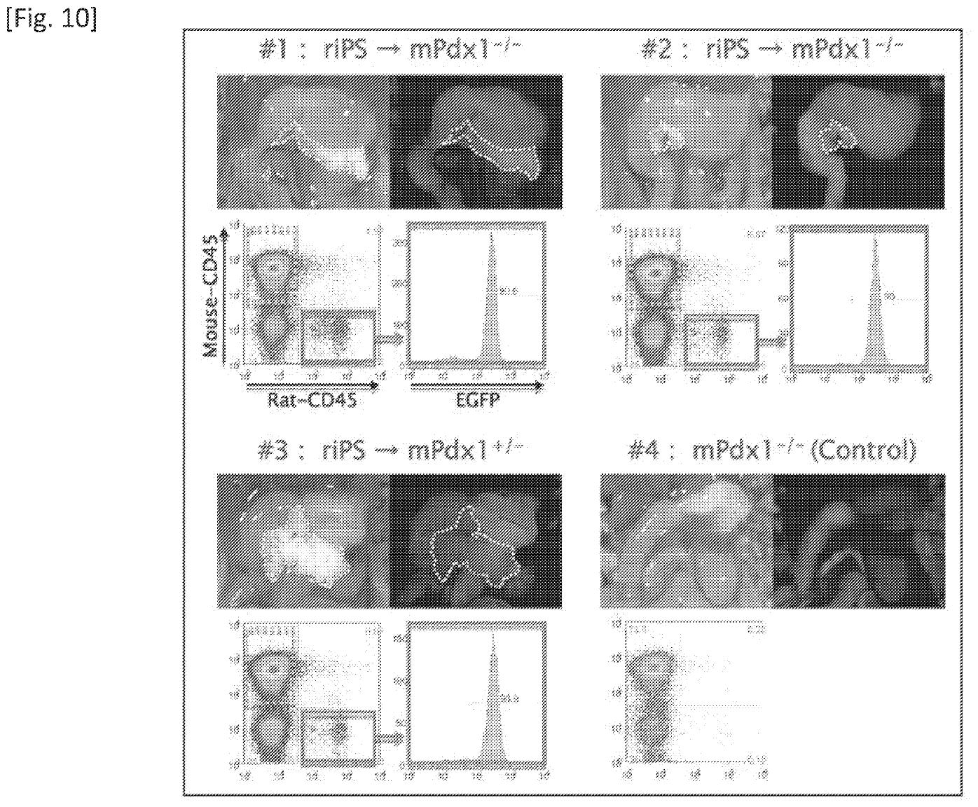

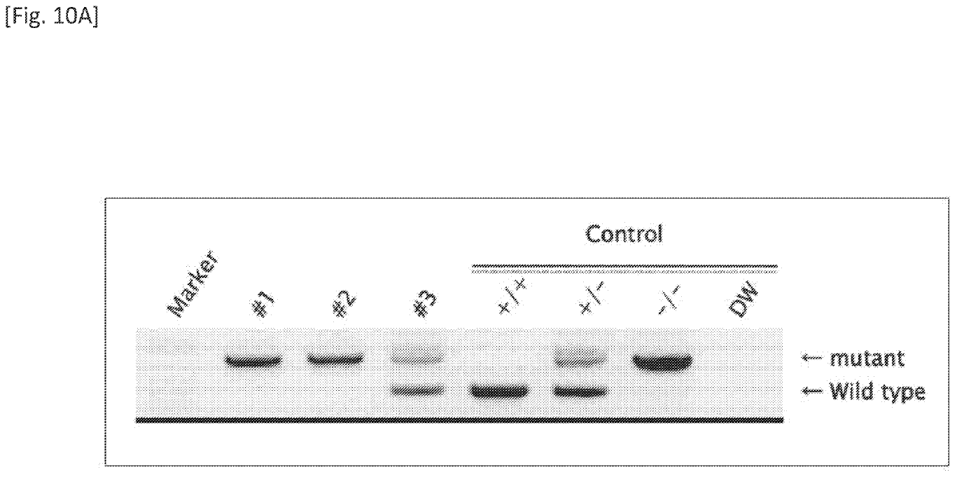

EGFP fluorescence was observed under a fluorescent stereoscopic microscope. It was found out from the EGFP expression on the body surface that neonate individual numbers #1, #2, and #3 were chimeras. By performing laparotomy thereon, pancreases uniformly expressing EGFP were observed in #1 and #2. Meanwhile, the pancreas of #3 exhibited partial EGFP expression, however, in a mosaic manner. Although #4 was a litter-mate as #1 to 3, no EGFP fluorescence was observed on the body surface. Because the pancreas was deficient upon laparotomy, it was found that #4 was a non-chimeric Pdx1 (−/−) mouse (FIG. 10).

Further, the spleens were removed from these neonates, and hemocyte cells prepared therefrom were subjected to staining with a monoclonal antibody against mouse or rat CD45, and analyzed by a flow cytometer. As a result, in the individual numbers #1 to 3, rat CD45-positive cells were observed in addition to mouse CD45-positive cells. Thus, it was confirmed that these were xenogeneic chimeric individuals between mouse and rat containing cells derived from the host mouse and the rat iPS cells. Furthermore, almost all the cells in the rat CD45-positive cell fractions exhibited EGFP fluorescence. Thus, the rat CD45-positive cells were cells derived from the rat iPS cells marked with EGFP (FIG. 10).

Moreover, as an experiment for confirming the basis of establishment of blastocyst complementation (the basis is vacancy of an organ (i.e., knockout (KO)), and what was confirmed was this), and to confirm that the genotype of the host mouse of the individual numbers #1 to #3 was KO by genotyping on a single cell for sure, mouse CD45-positive cells were collected from the spleen samples which had been analyzed by the flow cytometer, and genomic DNA was extracted and used for the genotyping.

The primers used for the genotyping were as follows:

Forward primer for identification of cell derived from injected embryo:

Common in mutant and wild type:
(SEQ ID NO: 16)
ATT GAG ATG AGA ACC GGC ATG
Reverse primer for identification of cell derived
from injected embryo:
For detection of mutant:
(SEQ ID NO: 17)
TTC AAC ATC ACT GCC AGC TCC
For detection of wild type:
(SEQ ID NO: 18)
TGT GAG CGA GTA ACA ACC

As a result, in #1 and #2, only mutant bands were observed, and in the individual number #3, both bands of mutant and wild type were detected. Accordingly, it was found that the genotype of the host mouse was Pdx1 (−/−) for #1, #2 and Pdx1 (+/−) for the individual number #3 (FIG. 10A). From this result, a pancreas of rat was successfully constructed in a mouse individual by applying the xenogeneic blastocyst complementation technique using the rat iPS cells as a donor in the Pdx1 (−/−) mice #1 and #2 which should not originally have pancreases formed.

### Example 6: Example of Using Animals Other Than Mouse

In the present example, it is demonstrated that organs can be produced even in the case of using animals other than mice. In regard to species other than mice, pluripotent stem cells having an ability to form a chimera can be established, similarly to Example 1, by producing iPS cells in accordance with the above preparation example to produce chimeras.

Here, iPS cells can also be produced in, for example, rat, pig, cattle, and human instead of mice, in accordance with Example 1.

For example, in the present example, it is conceived that similar experiments can also be carried out, by taking Example 1 into consideration, on animal species (rat (transgenic), pig (transgenic, knockout), and cattle (transgenic, knockout)) which are considered to be applicable to production of genetically modified animals, as the example other than mouse.

By this, it is possible to produce founder rat, pig, cattle, and the like, which are modified to have a lethal gene.

As described above, in the cases of using a rat, a pig, and cattle, similar experiments can be carried out as well in accordance with Example 1.

As described above, the present invention has been illustrated using preferred embodiments of the present invention, but it is understood that the scope of the present invention should be construed only by the claims. It is understood that the patents, patent applications, and articles cited herein should be such that the disclosures thereof should be incorporated into the present description by reference, as with the disclosures themselves are specifically described in the present description.

Sequence Listing Free Text

SEQ ID NO: 1: Forward primer for Oct3/4, Fw
(mOct3/4-S1120):
CCC TGG GGA TGC TGT GAG CCA AGG
SEQ ID NO: 2: Reverse primer for Oct3/4, Rv
(pMX/L3205):
CCC TTT TTC TGG AGA CTA AAT AAA
SEQ ID NO: 3: Forward primer for Klf4, Fw
(Klf4-S1236):
GCG AAC TCA CAC AGG CGA GAA ACC
SEQ ID NO: 4: Reverse primer for Klf4, Sox2 and
c-Myc, Rv (pMXs-AS3200):
TTA TCG TCG ACC ACT GTG CTG CTG
SEQ ID NO: 5: Forward primer for Sox2, Fw
(Sox2-S768):
ggt TAC CTC TTC CTC CCA CTC CAG
SEQ ID NO: 6: Forward primer for c-Myc, FW
(c-Myc-S1093):
CAG AGG AGG AAC GAG CTG AAG CGC
SEQ ID NO: 7 Forward (Fw) primer for
identification of cell derived injected embryo:
ATT GAG ATG AGA ACC GGC ATG
SEQ ID NO: 8 Reverse 1 (Rv1) primer for
identification of cell derived from injected
embryo:
TTC AAC ATC ACT GCC AGC TCC
SEQ ID NO: 9 Reverse (Rv2) primer for
identification of cell derived from injected
embryo:
TGT GAG CGA GTA ACA ACC
SEQ ID NO: 10 Forward (Fw) primer for detection
of transgene:
TGA CTT TCT GTG CTC AGA GG
SEQ ID NO: 11 Reverse (Rv) primer for detection
of transgene:
CAA TGA TGG CTC CAG GGT AA
SEQ ID NO: 12 Forward primer for detection of
cell (mutant) derived from injected embryo:
AAG GGA CTG GCT GCT ATT GG
SEQ ID NO: 13 Forward primer for detection of
cell (wild type) derived from injected embryo:
GTA CAC GTT TCT CCT CAG GAC
SEQ ID NO: 14 Reverse primer for cell (mutant)
derived from injected embryo:
ATA TCA CGG GAT GCC AAC GC
SEQ ID NO: 15 Reverse primer for detection of
cell (wild type) derived from injected embryo:
TCT CCA GTG TGA GTT CTC TCG
SEQ ID NO: 16 Forward primer (common in mutant
and wild type) for detection of cell derived
from injected embryo:
ATT GAG ATG AGA ACC GGC ATG
SEQ ID NO: 17 Reverse primer for detection of
cell (mutant) derived from injected embryo:
TTC AAC ATC ACT GCC AGC TCC
SEQ ID NO: 18 Reverse primer for detection of
cell (wild type) derived from injected embryo:
TGT GAG CGA GTA ACA ACC

1. A method for producing a target organ in a living body of a non-human mammal having an abnormality associated with a lack of development of the target organ in a development stage, the target organ produced being derived from a different individual mammal that is an individual different from the non-human mammal, the method comprising the steps:
a) preparing an induced pluripotent stem cell (iPS cell) derived from the different individual mammal;
b) transplanting the cell into a blastocyst stage fertilized egg of the non-human mammal;
c) developing the fertilized egg in a womb of a non-human surrogate parent mammal to obtain a litter; and
d) obtaining the target organ from the litter individual.

2. The method according to claim 1, wherein the iPS cell is derived from any one of a human, a rat, and a mouse.

3. The method according to claim 1, wherein the iPS cell is derived from any one of a rat and a mouse.

4. The method according to claim 1, wherein the organ to be produced is selected from a pancreas, a kidney, a thymus, and a hair.

5. The method according to claim 1, wherein the non-human mammal is a mouse.

6. The method according to claim 5, wherein the mouse is any one of a Sall1 knockout mouse, a Pdx1-Hes1 transgenic mouse, a Pdx1 knockout mouse, and a nude mouse.

7. The method according to claim 1, wherein the target organ is completely derived from the different individual mammal.

8. The method according to claim 1, further comprising a step of bringing a reprogramming factor into contact with a somatic cell to obtain the iPS cell.

9. The method according to claim 1, wherein the iPS cell and the non-human mammal are in a xenogeneic relationship.

10. The method according to claim 1, wherein the iPS cell is derived from a rat, and the non-human mammal is a mouse.

11. A non-human mammal having an abnormality associated with a lack of development of a target organ in a development stage, the mammal being produced by a method including the steps of:
a) preparing an iPS cell derived from a different individual mammal that is an individual different from the non-human mammal;
b) transplanting the iPS cell into a blastocyst stage fertilized egg of the non-human mammal; and
c) developing the fertilized egg in a womb of a non-human surrogate parent mammal to obtain a litter.

12. Use of a non-human mammal having an abnormality associated with a lack of development of a target organ in a development stage, for production of the target organ using an iPS cell.

13. A set for producing a target organ, the set comprising:
A) a non-human mammal having an abnormality associated with a lack of development of the target organ in a development stage; and
B) any one of
an iPS cell derived from a different individual mammal that is an individual different from the non-human mammal, and
a reprogramming factor and, if necessary, a somatic cell.

14. A method for producing any one of a target organ and a target body part, the method comprising the steps of:
A) providing an animal which includes a deficiency responsible gene coding for a factor which causes a deficiency of any one of an organ and a body part and gives any one of no possibility of survival and difficulty in survival if the factor functions, and in which the any one of an organ and a body part is complemented by blastocyst complementation, the deficiency responsible gene coding for a factor which causes a deficiency of the any one of a target organ and a target body part;
B) growing an ovum obtained from the animal into a blastocyst;
C) introducing a target iPS cell into the blastocyst so as to produce a chimeric blastocyst, the target iPS cell having a desired genome capable of complementing a deficiency caused by the deficiency responsible gene; and
D) producing an individual from the chimeric blastocyst, and then obtaining the any one of a target organ and a target body part from the individual.

15. The method according to claim 14, further comprising a step of bringing a reprogramming factor into contact with a somatic cell to obtain the iPS cell.

16. The method according to claim 14, wherein
the step D) includes developing the chimeric blastocyst in a womb of a non-human surrogate parent mammal to obtain a litter, and obtaining the target organ from the litter individual.

17. The method according to claim 14, wherein the target iPS cell is derived from any one of a rat and a mouse.

18. The method according to claim 14, wherein the any one of a target organ and a target body part is selected from a pancreas, a kidney, a thymus, and a hair.

19. The method according to claim 14, wherein the animal is a mouse.

20. The method according to claim 19, wherein the mouse is any one of a Sall1 knockout mouse, a Pdx1 knockout mouse, a Pdx1-Hes1 transgenic mouse, and a nude mouse.

21. The method according to claim 14, wherein the any one of a target organ and a target body part is completely derived from the target pluripotent cell.

22. The method according to claim 14, wherein the iPS cell and the non-human mammal are in a xenogeneic relationship.

23. The method according to claim 14, wherein the iPS cell is derived from a rat, and the non-human mammal is a mouse.

24. A set for producing any one of a target organ and a target body part, the set comprising:
A) a non-human animal which includes a gene coding for a factor which causes a deficiency of any one of an organ and a body part and gives any one of no possibility of survival and difficulty in survival if the factor functions, and in which the any one of an organ and a body part is complemented by complement; and
B) any one of
an iPS cell derived from a different individual mammal that is an individual different from the non-human mammal, and
a combination of a reprogramming factor and, if necessary, a somatic cell.

25. The set according to claim 24, wherein the non-human animal and the iPS cell are in a xenogeneic relationship.
